SEQUENCE LISTING (12 sequences)

SEQ ID NO: 1 - DNA, 2001 bp, Streptococcus thermophilus
  misc_feature 1..2001: note = ABC transporter permease DSM22587
  source 1..2001: mol_type = genomic DNA
atgtttcgtctaaccaatagactggctttgtccaatctcataaaaaatcgtaagctttactatccgtatacattggcaactattctggttattgccattacctatatttttacatctttaacgcttaattcgcatctggatgacttacctagggcagatgctatcaagacggttttggggttgggacttgggattgtcgccctatcctctgggattattgttctttatgccaacagctttgtcatgaaaaatcgttcaaaggagcttggtctctatagtgtgctaggcttagagaaacgtcacctctttagcatgattttaaaagaaacgatgattatgggttttgtgactttgcttctaggtataggtgtaggggcgctattcgataaactcatttatgcttttcttcaaaggcttatcggtgaaagtactggtttagtttcaacctttcaggtaatgacaattcctattgttcttgtcatctttgcttgtatttttagttttttggttttggtaaatggtttccgattgctacggcttaatcctttacaattaactaaagatggccttaagggtgaaaaaaagggacgtttcctagttattcagacctttttggggcttggggctatggggtatggttactatcttgctctttctgttcagaatccagtaatagctattatgagttttttcttggctgttttactagttattcttggaacttatcttctctttaatgccgggacaacagtagttctgcaactcttgaaaaagaagaaaagctactattacaagcctaataatatgatttccatttccaaccttgtctttcggatgaagaaaaatgcagttgggctggcgactattgctatcctctcaagtatggttttggtgacgcttgtaggagctgcaagtatctatgctgggaaaaaagactatttggtgagtgctgctccacatgattattcagtttcgggcaataaagttgaccttacaagcactaaaaagcttatggatgatttcttaatcaaaacaggtgagcaagtaaatgaagaagtagctgtctcttatcttttctttggtataaaaaatcaagaaactaataagttaaccgtttttactaaaaatgaaagaaaagttgttccaaaatcaattgttctggtcttctctcaagaaacctttaagcaattgacagggaaagaactcaatctcagttctaatcaaattgccttatacactaagaataaaacatttaagacgcaaaagagtctgtccatagatggtaagaactaccagattcataggcaacttggtgactttatcaataaaaaagtaccaaatatctataagattattgtgtcagattatagctacttagttgtaccagatatcaagatttttgagtcatcaatgaaagggacatcaatagcgcaagctacttatgttggtgttaacgtcaaggactccacacatgatgccaagaaaaatttagatttgctagaccaaatagcaggtgaagcaacaaaacagttagctggacaaactacaggtgtccctgagtcttacttctctgcaaacagccgatatgatgcagaagggatggtaaatggctttgttggcggaaccttctttattagtatcttcctatccatcatcttcatgcttggcacagttcttgtgatttactataaacagatttccgaaggttatgaggatcgtgagcgctttgttattcttcaaaagattggtttggacgaccttcaagtcaagcaaaccattcgtaagcaagttcttaccatcttcttcctccctttgatttttgcctttattcacttggcctttgcttatcatatgatcagtttgattgtgcgtatcattggagttctcaatccagatcttatgttagtggtcactatcattgtttgtggtgttttcttcctagcttatatcttggttttcgtcctaacatcgcgttcttatcgtagaatagtatcaatgtaa

SEQ ID NO: 2 - protein, 666 aa, Streptococcus thermophilus
  REGION 1..666: note = ABC transporter permease, DSM22587
MFRLTNRLALSNLIKNRKLYYPYTLATILVIAITYIFTSLTLNSHLDDLPRADAIKTVLGLGLGIVALSSGIIVLYANSFVMKNRSKELGLYSVLGLEKRHLFSMILKETMIMGFVTLLLGIGVGALFDKLIYAFLQRLIGESTGLVSTFQVMTIPIVLVIFACIFSFLVLVNGFRLLRLNPLQLTKDGLKGEKKGRFLVIQTFLGLGAMGYGYYLALSVQNPVIAIMSFFLAVLLVILGTYLLFNAGTTVVLQLLKKKKSYYYKPNNMISISNLVFRMKKNAVGLATIAILSSMVLVTLVGAASIYAGKKDYLVSAAPHDYSVSGNKVDLTSTKKLMDDFLIKTGEQVNEEVAVSYLFFGIKNQETNKLTVFTKNERKVVPKSIVLVFSQETFKQLTGKELNLSSNQIALYTKNKTFKTQKSLSIDGKNYQIHRQLGDFINKKVPNIYKIIVSDYSYLVVPDIKIFESSMKGTSIAQATYVGVNVKDSTHDAKKNLDLLDQIAGEATKQLAGQTTGVPESYFSANSRYDAEGMVNGFVGGTFFISIFLSIIFMLGTVLVIYYKQISEGYEDRERFVILQKIGLDDLQVKQTIRKQVLTIFFLPLIFAFIHLAFAYHMISLIVRIIGVLNPDLMLVVTIIVCGVFFLAYILVFVLTSRSYRRIVSM

SEQ ID NO: 3 - DNA, 2001 bp, Streptococcus thermophilus
  misc_feature 1..2001: note = ABC transporter permease DSM22933
  source 1..2001: mol_type = genomic DNA
atgtttcgtctaaccaatagactggctttgtccaatctcataaaaaatcgtaagctttactatccgtatacattggcaactattctggttattgccattacctatatttttacatctttaacgcttaattcgcatctggatgacttacctagggcagatgctatcaagacggttttggggttgggacttgggattgtcgccctatcctctgggattattgttctttatgccaacagctttgtcatgaaaaatcgttcaaaggagcttggtctctatagtgtgctaggcttagagaaacgtcacctctttagcatgattttaaaagaaacgatgattatgggttttgtgactttgcttctaggtataggtgtaggggcgctattcgataaactcatttatgcttttcttcaaaggcttatcggtgaaagtactggtttagtttcaacctttcaggtaatgacaattcctattgttcttgtcatctttgcttgtatttttagttttttggttttggtaaatggtttccgattgctacggcttaatcctttacaattaactaaagatggctttaagggtgaaaaaaagggacgtttcctagttattcagacctttttggggcttggggctatggggtatggttactatcttgctctttctgttcagaatccagtaatagctattatgagttttttcttggctgttttactagttattcttggaacttatcttctctttaatgccgggacaacagtagttctgcaactcttgaaaaagaagaaaagctactattacaagcctaataatatgatttccatttccaaccttgtctttcggatgaagaaaaatgcagttgggctggcgactattgctatcctctcaagtatggttttggtgacgcttgtaggagctgcaagtatctatgctgggaaaaaagactatttggtgagtgctgctccacatgattattcagtttcgggcaataaagttgaccttacaagcactaaaaagcttatggatgatttcttaatcaaaacaggtgagcaagtaaatgaagaagtagctgtctcttatcttttctttggtataaaaaatcaagaaactaataagttaaccgtttttactaaaaatgaaagaaaagttgttccaaaatcaattgttctggtcttctctcaagaaacctttaagcaattgacagggaaagaactcaatctcagttctaatcaaattgccttatacactaagaataaaacatttaagacgcaaaagagtctgtccatagatggtaagaactaccagattcataggcaacttggtgactttatcaataaaaaagtaccaaatatctataagattattgtgtcagattatagctacttagttgtaccagatatcaagatttttgagtcatcaatgaaagggacatcaatagcgcaagctacttatgttggtgttaacgtcaaggactccacacatgatgccaagaaaaatttagatttgctagaccaaatagcaggtgaagcaacaaaacagttagctggacaaactacaggtgtccctgagtcttacttctctgcaaacagccgatatgatgcagaagggatggtaaatggctttgttggcggaaccttctttattagtatcttcctatccatcatcttcatgcttggcacagttcttgtgatttactataaacagatttccgaaggttatgaggatcgtgagcgctttgttattcttcaaaagattggtttggacgaccttcaagtcaagcaaaccattcgtaagcaagttcttaccatcttcttcctccctttgatttttgcctttattcacttggcctttgcttatcatatgatcagtttgattgtgcgtatcattggagttctcaatccagatcttatgttagtggtcactatcattgtttgtggtgttttcttcctagcttatatcttggttttcgtcctaacatcgcgttcttatcgtagaatagtatcaatgtaa

SEQ ID NO: 4 - protein, 666 aa, Streptococcus thermophilus
  REGION 1..666: note = ABC transporter permease DSM22933
MFRLTNRLALSNLIKNRKLYYPYTLATILVIAITYIFTSLTLNSHLDDLPRADAIKTVLGLGLGIVALSSGIIVLYANSFVMKNRSKELGLYSVLGLEKRHLFSMILKETMIMGFVTLLLGIGVGALFDKLIYAFLQRLIGESTGLVSTFQVMTIPIVLVIFACIFSFLVLVNGFRLLRLNPLQLTKDGFKGEKKGRFLVIQTFLGLGAMGYGYYLALSVQNPVIAIMSFFLAVLLVILGTYLLFNAGTTVVLQLLKKKKSYYYKPNNMISISNLVFRMKKNAVGLATIAILSSMVLVTLVGAASIYAGKKDYLVSAAPHDYSVSGNKVDLTSTKKLMDDFLIKTGEQVNEEVAVSYLFFGIKNQETNKLTVFTKNERKVVPKSIVLVFSQETFKQLTGKELNLSSNQIALYTKNKTFKTQKSLSIDGKNYQIHRQLGDFINKKVPNIYKIIVSDYSYLVVPDIKIFESSMKGTSIAQATYVGVNVKDSTHDAKKNLDLLDQIAGEATKQLAGQTTGVPESYFSANSRYDAEGMVNGFVGGTFFISIFLSIIFMLGTVLVIYYKQISEGYEDRERFVILQKIGLDDLQVKQTIRKQVLTIFFLPLIFAFIHLAFAYHMISLIVRIIGVLNPDLMLVVTIIVCGVFFLAYILVFVLTSRSYRRIVSM

SEQ ID NO: 5 - DNA, 615 bp, Streptococcus thermophilus
  misc_feature 1..615: note = Peptide deformylase DSM22587
  source 1..615: mol_type = genomic DNA
atggatgctcaaaccaaaattattcgcgccagccacatgattgatatgaacgatatcatacgcgaaggcaacccaaccttgcgtgctgtcgctgaagacgtaaccctaccactttcagatgaagatattatccttggtgaaaaaatgatgcagtttcttcgtaattcacaggaccctgttatcgctgaaaaaatgggacttcgaggaggtgttggtcttgcagcaccacaattagatatttcaaaacgcattattgctgttctcgttccaaatcctgaagacgctaaggggaatccacctaaagaagcttatagccttcaagaaatcatgtataatcctaaagtagttgctcattctgttcaggaggctgctctaggtaacggtgaaggatgcctttcagtcgatcgcgacgttcctggatatgtcgttcgccatgctcgtgttactattgaatacttcaacaaagagggtgaaaagaaacgtattaaactccgtggttacgactcaatcgttgttcaacatgaaatcgaccatactaacggtatcatgttctacgaccgtatcaataaagacaatccatttactatcaaggatggactcttgattatcgaataa

SEQ ID NO: 6 - protein, 204 aa, Streptococcus thermophilus
  REGION 1..204: note = Peptide deformylase DSM22587
MDAQTKIIRASHMIDMNDIIREGNPTLRAVAEDVTLPLSDEDIILGEKMMQFLRNSQDPVIAEKMGLRGGVGLAAPQLDISKRIIAVLVPNPEDAKGNPPKEAYSLQEIMYNPKVVAHSVQEAALGNGEGCLSVDRDVPGYVVRHARVTIEYFNKEGEKKRIKLRGYDSIVVQHEIDHTNGIMFYDRINKDNPFTIKDGLLIIE

SEQ ID NO: 7 - DNA, 615 bp, Streptococcus thermophilus
  misc_feature 1..615: note = Peptide deformylase DSM22933
  source 1..615: mol_type = genomic DNA
atggatgctcaaaccaaaattattcgcgccagccacatgattgatatgaacgatatcatacgcgaaggcaacccaaccttgcgtgctgtcgctgaagacgtaaccctaccactttcagatgaagatattatccttggtgaaaaaatgatgcagtttcttcgtaattcacaggaccctgttatcgctgaaaaaatgggacttcgaggaggtgttggtcttgcagcaccacaattagatatttcaaaacgcattattgctgttctcgttccaaatcctgaagacgctaaggggaatccacctaaagaagcttatagccttcaagaaatcatgtataatcctaaagtagttgctcattctgttcaggaggctgctctaggtaacggtgaaggatgcctttcagtcgatcgcgacgttcctggatatgtcgtttgccatgctcgtgttactattgaatacttcaacaaagagggtgaaaagaaacgtattaaactccgtggttacgactcaatcgttgttcaacatgaaatcgaccatactaacggtatcatgttctacgaccgtatcaataaagacaatccatttactatcaaggatggactcttgattatcgaataa

SEQ ID NO: 8 - protein, 204 aa, Streptococcus thermophilus
  REGION 1..204: note = Peptide deformylase DSM22933
MDAQTKIIRASHMIDMNDIIREGNPTLRAVAEDVTLPLSDEDIILGEKMMQFLRNSQDPVIAEKMGLRGGVGLAAPQLDISKRIIAVLVPNPEDAKGNPPKEAYSLQEIMYNPKVVAHSVQEAALGNGEGCLSVDRDVPGYVVCHARVTIEYFNKEGEKKRIKLRGYDSIVVQHEIDHTNGIMFYDRINKDNPFTIKDGLLIIE

SEQ ID NO: 9 - DNA, 765 bp, Streptococcus thermophilus
  misc_feature 1..765: note = Branched-chain amino acid transport ATP-binding protein LivGDSM22587
  source 1..765: mol_type = genomic DNA
atggcacttcttgaagttaaaaatttaactaaaaactttggtggtttgactgctgttggtgatgtttcaatggaactcaatgaaggtgagttggttgggctaatagggccaaacggtgctggtaaaacaaccttgttcaaccttttgactggtgtctatgagccaagtgaagggactgtaacgcttgatggtatagttctcaacggtaaagcaccttacaagattgcgtcactcggtttgtcacgtactttccaaaatatccgccttttcaaagacatgactgtacttgaaaatgttcttgttggtttatcaaataagcaaccttcaaatttctttgcatctcttttgcgcttgcctaagtactattcaagtgaggaagagttgaaagacaaagctatgaagctcttggctatctttaacttggatggtgaggcagatacgcttgcgaaaaacttggcttatggacaacaacgtcacttggagattgttcgtgcgcttgcaacggaacctaaaattcttttcctcgatgaaccagctgctggtatgaacccacaagaaacagctgagttgactgctcgtattcgtcaaattcaaaaagatttcggtattacaattatcttgattgagcacgatatgagtttggtcatggatgtcactgagcgtatctatgttttagaatatggacgcttgattgcagaaggaacccctgatgaaattaagaataacaagcgtgttatcgaagcttacttgggaggtgaagcataa

SEQ ID NO: 10 - protein, 254 aa, Streptococcus thermophilus
  REGION 1..254: note = Branched-chain amino acid transport ATP-binding protein LivGDSM22587
MALLEVKNLTKNFGGLTAVGDVSMELNEGELVGLIGPNGAGKTTLFNLLTGVYEPSEGTVTLDGIVLNGKAPYKIASLGLSRTFQNIRLFKDMTVLENVLVGLSNKQPSNFFASLLRLPKYYSSEEELKDKAMKLLAIFNLDGEADTLAKNLAYGQQRHLEIVRALATEPKILFLDEPAAGMNPQETAELTARIRQIQKDFGITIILIEHDMSLVMDVTERIYVLEYGRLIAEGTPDEIKNNKRVIEAYLGGEA

SEQ ID NO: 11 - DNA, 765 bp, Streptococcus thermophilus
  misc_feature 1..765: note = Branched-chain amino acid transport ATP-binding protein LivGDSM22933
  source 1..765: mol_type = genomic DNA
atggcacttcttgaagttaaaaatttaactaaaaactttggtggtttgactgctgttggtgatgtttcaatggaactcaatgaaggtgagttggttgggctaatagggccaaacggtgctggtaaaacaaccttgttcaaccttttgactggtgtctatgagccaagtgaagggactgtaacgcttgatggtatagttctcaacggtaaagcaccttacaagattgcgtcactcggtttgtcacgtactttccaaaatatccgccttttcaaagacatgactgtacttgaaaatgttcttgttggtttatcaaataagcaaccttcaaatttctttgcatctcttttgcgcttgcctaagtactattcaagtgaggaagagttgaaagacaaagctatgaagctcttggctatctttaacttggatggtgaggcagatacgcttgcgaaaaacttggcttatggacaacaacgtcacttggagattgttcgtgcgcttgcaacggtacctaaaattcttttcctcgatgaaccagctgctggtatgaacccacaagaaacagctgagttgactgctcgtattcgtcaaattcaaaaagatttcggtattacaattatcttgattgagcacgatatgagtttggtcatggatgtcactgagcgtatctatgttttagaatatggacgcttgattgcagaaggaacccctgatgaaattaagaataacaagcgtgttatcgaagcttacttgggaggtgaagcataa

SEQ ID NO: 12 - protein, 254 aa, Streptococcus thermophilus
  REGION 1..254: note = Branched-chain amino acid transport ATP-binding protein LivGDSM22933
MALLEVKNLTKNFGGLTAVGDVSMELNEGELVGLIGPNGAGKTTLFNLLTGVYEPSEGTVTLDGIVLNGKAPYKIASLGLSRTFQNIRLFKDMTVLENVLVGLSNKQPSNFFASLLRLPKYYSSEEELKDKAMKLLAIFNLDGEADTLAKNLAYGQQRHLEIVRALATVPKILFLDEPAAGMNPQETAELTARIRQIQKDFGITIILIEHDMSLVMDVTERIYVLEYGRLIAEGTPDEIKNNKRVIEAYLGGEA

(19) United States
(12) Patent Application Publication
(10) Pub. No.: US 2025/0101372 A1
(43) Pub. Date: Mar. 27, 2025

(54) Streptococcus thermophilus Strains with Improved Texturing Properties

(71) Applicant: Chr. Hansen A/S, Hoersholm (DK)

(72) Inventors: Thomas JANZEN, Hoersholm (DK); Ditte Ellegaard CHRISTENSEN, Hoersholm (DK); Jesper BROEND, Hoersholm (DK); Jeorgos TRIHAAS, Hoersholm (DK); Stephanie SERRAULT, Hoersholm (DK)

(73) Assignee: Chr. Hansen A/S, Hoersholm (DK)

(21) Appl. No.: 18/728,307

(22) PCT Filed: Jan. 25, 2023
(86) PCT No.: PCT/EP2023/051744

§ 371 Date: Jul. 11, 2024

(30) Foreign Application Priority Data
Jan. 28, 2022 (EP) 22153930.7

(51) Int. Cl. C12N 1/20; A23C 9/123; C12N 9/80; C12Q 1/24; C12R 1/46

(52) U.S. Cl. CPC C12N 1/20; A23C 9/1238; C12N 9/80; C12Q 1/24; C12R 2001/46; C12Y 305/01088

(57) ABSTRACT

The invention provides Streptococcus thermophilus mutants, such as DSM22933, which have improved texturizing properties. The strains have: (i) a mutation in the branched chain amino acid transport ATP-binding protein LivG (TC 3.A.1.4.1) gene; (ii) a mutation in the ABC transporter permease protein gene; and/or (iii) a mutation in the peptide deformylase protein gene. The invention, furthermore, relates to compositions, such as a starter culture, comprising one or more of these mutants, to fermented products made using these mutants and to the use of the mutants and compositions of the invention.

### FIELD OF THE INVENTION

The present invention relates to Streptococcus thermophilus mutants, which have improved texturizing properties. The present invention, furthermore, relates to compositions, such as a starter culture, comprising one or more of these mutants, to fermented products made using these mutants and to the use of the mutants and compositions of the invention.

### BACKGROUND OF THE INVENTION

The food industry uses numerous bacteria, in particular lactic acid bacteria, in order to improve the texture of food. In the dairy industry, lactic acid bacteria are used intensively in order to bring about the acidification of milk (by fermentation) but also in order to texturize the product into which they are incorporated.

Among the lactic acid bacteria used in the food industry, Streptococcus, Lactococcus, Lactobacillus, Leuconostoc, Pediococcus and Bifidobacterium are predominantly applied. The lactic acid bacteria of the species Streptococcus thermophilus (S. thermophilus) are used extensively alone or in combination with other bacteria such as Lactobacillus delbrueckii subsp bulgaricus (L. bulgaricus) for the production of food products, in particular fermented food products. They are used in particular in the formulation of the ferments used for the production of fermented milks, for example yoghurt. Certain of them play a dominant role in the development of the texture of the fermented product. This characteristic is closely linked to the production of extracellular polymeric substances (exopolysaccharides, EPS) that are secreted by the lactic acid bacteria into the surrounding environment.

There is a desire of obtaining fermented milk products with high texture. For instance, the current trend in yoghurt production is aiming for mild flavor and high texture. Today this is achieved by the use of cultures which produce a mild flavor and the addition of thickeners or proteins to give the desired thickness. Producers of fermented products (such as yoghurt producers) would like to be able to make fermented products, such as yoghurt, with these properties without the addition of thickening agents. This will help them reduce cost and give a cleaner label. One very attractive way to achieve this would be to have a starter culture which produces a high level of texture.

Many strains of S. thermophilus synthesize extracellular polysaccharides (EPS). These molecules may be produced as capsules that are tightly associated with the cell, or they may be liberated into the medium as a loose slime (i.e., “ropy” polysaccharide). Although the presence of exopolysaccharide does not confer any obvious advantage to growth or survival of S. thermophilus in milk, in situ production by this species or other dairy lactic acid bacteria typically imparts a desirable “ropy” or viscous texture to fermented milk products. Work has also shown that exopolysaccharide-producing S. thermophilus can enhance the functional properties of fermented milk products. For further details see the review article of Broadbent et al. (J. Dairy Sci., 2003, 86:407-423).

In order to meet the requirements of the industry, it has become necessary to provide novel texturizing strains of lactic acid bacteria, in particular of S. thermophilus, for texturizing food products. Especially there is a need for novel texturizing strains of S. thermophilus which can be used together with texturizing strains of Lactobacillus such as, e.g., L. bulgaricus.

### SUMMARY OF THE INVENTION

The present invention provides novel S. thermophilus strains with improved properties in particular in relation to their ability to improve texture of fermented food products, like dairy products, such as, e.g., yogurt, as well as dairy analogue products, and which is useful in present-day highly industrialized fermented food production.

One aspect of the present invention relates to a S. thermophilus strain having (i) a mutation in the branched chain amino acid transport ATP-binding protein LivG (TC 3.A.1.4.1) gene, wherein the mutation is preferably a substitution of Glutamic acid to Valine at a position corresponding to position 169 of SEQ ID NO.: 12. In another aspect, the present invention relates to a S. thermophilus strain having a mutation in the branched chain amino acid transport ATP-binding protein LivG (TC 3.A.1.4.1) gene, wherein the mutation is preferably a substitution of nucleotide A to nucleotide T at a position corresponding to position 506 of SEQ ID NO.: 11.

In one aspect of the invention, the S. thermophilus strain has (ii) a mutation in the ABC transporter permease protein gene, wherein the mutation is preferably a substitution of Leucine to Phenylalanine at a position corresponding to position 190 of SEQ ID NO.: 4. In a further aspect, the S. thermophilus strain of the invention has an additional mutation in the ABC transporter permease protein gene, wherein the mutation is preferably a substitution of nucleotide C to nucleotide T at a position corresponding to position 568 of SEQ ID NO.: 3.

In one aspect of the invention, the S. thermophilus strain has a (iii) mutation in the peptide deformylase protein gene, wherein the mutation is preferably a substitution of Arginine to Cysteine at a position corresponding to position 144 of SEQ ID NO.: 8. In a further aspect, the S. thermophilus strain of the invention has a mutation in the peptide deformylase protein gene, wherein the mutation is preferably a substitution of nucleotide C to nucleotide T at a position corresponding to position 430 of SEQ ID NO.: 7.

In one aspect, the S. thermophilus strain has a (i) mutation in the branched chain amino acid transport ATP-binding protein LivG gene. In one aspect, the S. thermophilus strain has a (ii) mutation in the ABC transporter permease protein gene. In one aspect, the S. thermophilus strain has a (iii) mutation in the peptide deformylase protein gene. In another aspect, the S. thermophilus strain has two of the mutations as described above, such as mutations (i) and (ii), i.e., a mutation in the branched chain amino acid transport ATP-binding protein LivG gene and in the ABC transporter permease protein gene; such as mutations (i) and (iii), as described above, or such as mutations (ii) and (iii), as described above. In a preferred aspect, the S. thermophilus strain has all three mutations (i), (ii), and (iii) as defined above.

Hence, the present invention relates to a S. thermophilus strain having (i) a Valine at a position corresponding to position 169 of SEQ ID NO.: 12 and/or a T at a position corresponding to position 506 of SEQ ID NO.: 11 (the branched chain amino acid transport ATP-binding protein LivG (TC 3.A.1.4.1) gene). The present invention relates to a S. thermophilus strain having (ii) a Phenylalanine at a position corresponding to position 190 of SEQ ID NO.: 4 and/or a T at a position corresponding to position 568 of SEQ ID NO.: 3 (ABC transporter permease protein gene). The present invention relates to a S. thermophilus strain having (iii) a Cysteine at a position corresponding to position 144 of SEQ ID NO.: 8 and/or a T at a position corresponding to position 430 of SEQ ID NO.: 7 (peptide deformylase protein gene). In a preferred aspect, the branched chain amino acid transport ATP-binding protein LivG (TC 3.A.1.4.1) gene of the S. thermophilus strain of the invention has a T at a position corresponding to position 506 of SEQ ID NO.: 11, and/or the branched chain amino acid transport ATP-binding protein LivG (TC 3.A.1.4.1) has a valine at a position corresponding to position 169 of SEQ ID NO.: 12. In a further preferred aspect, the ABC transporter permease protein gene of the S. thermophilus strain of the invention has a T at a position corresponding to position 568 of SEQ ID NO.: 3 and/or the ABC transporter permease protein has a Phenylalanine at a position corresponding to position 190 of SEQ ID NO.: 4. In another aspect, the S. thermophilus strain of the invention has both (i) and (ii) as described above. In another aspect, the S. thermophilus strain of the invention has (i) and (iii) as described above. In a further aspect, the S. thermophilus strain of the invention has (ii) and (iii) as described above. In a preferred aspect, the S. thermophilus strain of the invention has (i), (ii) and (iii) as described above.

Preferably, the S. thermophilus strain is DSM22933 or a mutant or variant thereof, preferably wherein the mutant or variant shows the same or similar texturing properties as DSM22933.

In one aspect the present invention relates to compositions comprising said S. thermophilus strain.

In one aspect the present invention relates to methods of producing a fermented product comprising fermenting a substrate with the S. thermophilus strain or the compositions of the invention.

In one aspect the present invention relates to fermented food product obtainable by the method according to the invention or comprising a S. thermophilus strain or comprising a composition according to the invention.

The present invention also relates to methods for manufacturing S. thermophilus strains as defined by the invention; wherein the strains are preferably obtained by using the strain DSM22587 as a starting material i.e. as a mother strain.

The present invention further relates to the use of the S. thermophilus strain of the invention, or the composition according to the invention for the manufacture of a fermented product, preferably for the manufacture of a fermented milk product.

### SEQUENCE LISTING

SEQ ID NO:1 sets out the nucleotide sequence of the ABC transporter permease of DSM22587.

SEQ ID NO.:2 sets out the amino acid sequence of the ABC transporter permease of DSM22587.

SEQ ID NO.:3 sets out the nucleotide sequence of the ABC transporter permease of DSM22933.

SEQ ID NO.:4 sets out the amino acid sequence of the ABC transporter permease of DSM22933.

SEQ ID NO.:5 sets out the nucleotide sequence of the Peptide deformylase of DSM22587.

SEQ ID NO.:6 sets out the amino acid sequence of the Peptide deformylase of DSM22587.

SEQ ID NO.:7 sets out the nucleotide sequence of the Peptide deformylase of DSM22933.

SEQ ID NO.:8 sets out the amino acid sequence of the Peptide deformylase of DSM22933.

SEQ ID NO.:9 sets out the nucleotide sequence of the branched-chain amino acid transport ATP-binding protein LivG of DSM22587.

SEQ ID NO.:10 sets out the amino acid sequence of the branched-chain amino acid transport ATP-binding protein LivG of DSM22587.

SEQ ID NO.:11 sets out the nucleotide sequence of the branched-chain amino acid transport ATP-binding protein LivG of DSM22933.

SEQ ID NO.:12 sets out the amino acid sequence of the branched-chain amino acid transport ATP-binding protein LivG of DSM22933.

ABC transporter permease, DSM22587
DNA sequence, 2001 bps
SEQ ID NO.: 1
ATGTTTCGTCTAACCAATAGACTGGCTTTGTCCAATCTCATAAAAAATCGTAAGCTTTACTATCCGTAT
ACATTGGCAACTATTCTGGTTATTGCCATTACCTATATTTTTACATCTTTAACGCTTAATTCGCATCTGG
ATGACTTACCTAGGGCAGATGCTATCAAGACGGTTTTGGGGTTGGGACTTGGGATTGTCGCCCTATC
CTCTGGGATTATTGTTCTTTATGCCAACAGCTTTGTCATGAAAAATCGTTCAAAGGAGCTTGGTCTCTA
TAGTGTGCTAGGCTTAGAGAAACGTCACCTCTTTAGCATGATTTTAAAAGAAACGATGATTATGGGTT
TTGTGACTTTGCTTCTAGGTATAGGTGTAGGGGCGCTATTCGATAAACTCATTTATGCTTTTCTTCAAA
GGCTTATCGGTGAAAGTACTGGTTTAGTTTCAACCTTTCAGGTAATGACAATTCCTATTGTTCTTGTCA
TCTTTGCTTGTATTTTTAGTTTTTTGGTTTTGGTAAATGGTTTCCGATTGCTACGGCTTAATCCTTTACA
ATTAACTAAAGATGGCCTTAAGGGTGAAAAAAAGGGACGTTTCCTAGTTATTCAGACCTTTTTGGGG
CTTGGGGCTATGGGGTATGGTTACTATCTTGCTCTTTCTGTTCAGAATCCAGTAATAGCTATTATGAG
TTTTTTCTTGGCTGTTTTACTAGTTATTCTTGGAACTTATCTTCTCTTTAATGCCGGGACAACAGTAGTT
CTGCAACTCTTGAAAAAGAAGAAAAGCTACTATTACAAGCCTAATAATATGATTTCCATTTCCAACCTT
GTCTTTCGGATGAAGAAAAATGCAGTTGGGCTGGCGACTATTGCTATCCTCTCAAGTATGGTTTTGGT
GACGCTTGTAGGAGCTGCAAGTATCTATGCTGGGAAAAAAGACTATTTGGTGAGTGCTGCTCCACAT
GATTATTCAGTTTCGGGCAATAAAGTTGACCTTACAAGCACTAAAAAGCTTATGGATGATTTCTTAAT
CAAAACAGGTGAGCAAGTAAATGAAGAAGTAGCTGTCTCTTATCTTTTCTTTGGTATAAAAAATCAAG
AAACTAATAAGTTAACCGTTTTTACTAAAAATGAAAGAAAAGTTGTTCCAAAATCAATTGTTCTGGTC
TTCTCTCAAGAAACCTTTAAGCAATTGACAGGGAAAGAACTCAATCTCAGTTCTAATCAAATTGCCTT
ATACACTAAGAATAAAACATTTAAGACGCAAAAGAGTCTGTCCATAGATGGTAAGAACTACCAGATT
CATAGGCAACTTGGTGACTTTATCAATAAAAAAGTACCAAATATCTATAAGATTATTGTGTCAGATTA
TAGCTACTTAGTTGTACCAGATATCAAGATTTTTGAGTCATCAATGAAAGGGACATCAATAGCGCAA
GCTACTTATGTTGGTGTTAACGTCAAGGACTCCACACATGATGCCAAGAAAAATTTAGATTTGCTAGA
CCAAATAGCAGGTGAAGCAACAAAACAGTTAGCTGGACAAACTACAGGTGTCCCTGAGTCTTACTTC
TCTGCAAACAGCCGATATGATGCAGAAGGGATGGTAAATGGCTTTGTTGGCGGAACCTTCTTTATTA
GTATCTTCCTATCCATCATCTTCATGCTTGGCACAGTTCTTGTGATTTACTATAAACAGATTTCCGAAG
GTTATGAGGATCGTGAGCGCTTTGTTATTCTTCAAAAGATTGGTTTGGACGACCTTCAAGTCAAGCAA
ACCATTCGTAAGCAAGTTCTTACCATCTTCTTCCTCCCTTTGATTTTTGCCTTTATTCACTTGGCCTTTGC
TTATCATATGATCAGTTTGATTGTGCGTATCATTGGAGTTCTCAATCCAGATCTTATGTTAGTGGTCAC
TATCATTGTTTGTGGTGTTTTCTTCCTAGCTTATATCTTGGTTTTCGTCCTAACATCGCGTTCTTATCGT
AGAATAGTATCAATGTAA
AA sequence, 666 amino acids
SEQ ID NO.: 2
MFRLTNRLALSNLIKNRKLYYPYTLATILVIAITYIFTSLTLNSHLDDLPRADAIKTVLGLGLGIVALSSGIIVLYA
NSFVMKNRSKELGLYSVLGLEKRHLFSMILKETMIMGFVTLLLGIGVGALFDKLIYAFLQRLIGESTGLVSTF
QVMTIPIVLVIFACIFSFLVLVNGFRLLRLNPLQLTKDGLKGEKKGRFLVIQTFLGLGAMGYGYYLALSVQNP
VIAIMSFFLAVLLVILGTYLLFNAGTTVVLQLLKKKKSYYYKPNNMISISNLVFRMKKNAVGLATIAILSSMVL
VTLVGAASIYAGKKDYLVSAAPHDYSVSGNKVDLTSTKKLMDDFLIKTGEQVNEEVAVSYLFFGIKNQETN
KLTVFTKNERKVVPKSIVLVFSQETFKQLTGKELNLSSNQIALYTKNKTFKTQKSLSIDGKNYQIHRQLGDFI
NKKVPNIYKIIVSDYSYLVVPDIKIFESSMKGTSIAQATYVGVNVKDSTHDAKKNLDLLDQIAGEATKQLAG
QTTGVPESYFSANSRYDAEGMVNGFVGGTFFISIFLSIIFMLGTVLVIYYKQISEGYEDRERFVILQKIGLDDL
QVKQTIRKQVLTIFFLPLIFAFIHLAFAYHMISLIVRIIGVLNPDLMLVVTIIVCGVFFLAYILVFVLTSRSYRRIV
SM
ABC transporter permease, DSM22933
DNA sequence, 2001 bps
SEQ ID NO.: 3
ATGTTTCGTCTAACCAATAGACTGGCTTTGTCCAATCTCATAAAAAATCGTAAGCTTTACTATCCGTAT
ACATTGGCAACTATTCTGGTTATTGCCATTACCTATATTTTTACATCTTTAACGCTTAATTCGCATCTGG
ATGACTTACCTAGGGCAGATGCTATCAAGACGGTTTTGGGGTTGGGACTTGGGATTGTCGCCCTATC
CTCTGGGATTATTGTTCTTTATGCCAACAGCTTTGTCATGAAAAATCGTTCAAAGGAGCTTGGTCTCTA
TAGTGTGCTAGGCTTAGAGAAACGTCACCTCTTTAGCATGATTTTAAAAGAAACGATGATTATGGGTT
TTGTGACTTTGCTTCTAGGTATAGGTGTAGGGGCGCTATTCGATAAACTCATTTATGCTTTTCTTCAAA
GGCTTATCGGTGAAAGTACTGGTTTAGTTTCAACCTTTCAGGTAATGACAATTCCTATTGTTCTTGTCA
TCTTTGCTTGTATTTTTAGTTTTTTGGTTTTGGTAAATGGTTTCCGATTGCTACGGCTTAATCCTTTACA
ATTAACTAAAGATGGCTTTAAGGGTGAAAAAAAGGGACGTTTCCTAGTTATTCAGACCTTTTTGGGG
CTTGGGGCTATGGGGTATGGTTACTATCTTGCTCTTTCTGTTCAGAATCCAGTAATAGCTATTATGAG
TTTTTTCTTGGCTGTTTTACTAGTTATTCTTGGAACTTATCTTCTCTTTAATGCCGGGACAACAGTAGTT
CTGCAACTCTTGAAAAAGAAGAAAAGCTACTATTACAAGCCTAATAATATGATTTCCATTTCCAACCTT
GTCTTTCGGATGAAGAAAAATGCAGTTGGGCTGGCGACTATTGCTATCCTCTCAAGTATGGTTTTGGT
GACGCTTGTAGGAGCTGCAAGTATCTATGCTGGGAAAAAAGACTATTTGGTGAGTGCTGCTCCACAT
GATTATTCAGTTTCGGGCAATAAAGTTGACCTTACAAGCACTAAAAAGCTTATGGATGATTTCTTAAT
CAAAACAGGTGAGCAAGTAAATGAAGAAGTAGCTGTCTCTTATCTTTTCTTTGGTATAAAAAATCAAG
AAACTAATAAGTTAACCGTTTTTACTAAAAATGAAAGAAAAGTTGTTCCAAAATCAATTGTTCTGGTC
TTCTCTCAAGAAACCTTTAAGCAATTGACAGGGAAAGAACTCAATCTCAGTTCTAATCAAATTGCCTT
ATACACTAAGAATAAAACATTTAAGACGCAAAAGAGTCTGTCCATAGATGGTAAGAACTACCAGATT
CATAGGCAACTTGGTGACTTTATCAATAAAAAAGTACCAAATATCTATAAGATTATTGTGTCAGATTA
TAGCTACTTAGTTGTACCAGATATCAAGATTTTTGAGTCATCAATGAAAGGGACATCAATAGCGCAA
GCTACTTATGTTGGTGTTAACGTCAAGGACTCCACACATGATGCCAAGAAAAATTTAGATTTGCTAGA
CCAAATAGCAGGTGAAGCAACAAAACAGTTAGCTGGACAAACTACAGGTGTCCCTGAGTCTTACTTC
TCTGCAAACAGCCGATATGATGCAGAAGGGATGGTAAATGGCTTTGTTGGCGGAACCTTCTTTATTA
GTATCTTCCTATCCATCATCTTCATGCTTGGCACAGTTCTTGTGATTTACTATAAACAGATTTCCGAAG
GTTATGAGGATCGTGAGCGCTTTGTTATTCTTCAAAAGATTGGTTTGGACGACCTTCAAGTCAAGCAA
ACCATTCGTAAGCAAGTTCTTACCATCTTCTTCCTCCCTTTGATTTTTGCCTTTATTCACTTGGCCTTTGC
TTATCATATGATCAGTTTGATTGTGCGTATCATTGGAGTTCTCAATCCAGATCTTATGTTAGTGGTCAC
TATCATTGTTTGTGGTGTTTTCTTCCTAGCTTATATCTTGGTTTTCGTCCTAACATCGCGTTCTTATCGT
AGAATAGTATCAATGTAA
AA sequence, 666 amino acids
SEQ ID NO.: 4
MFRLTNRLALSNLIKNRKLYYPYTLATILVIAITYIFTSLTLNSHLDDLPRADAIKTVLGLGLGIVALSSGIIVLYA
NSFVMKNRSKELGLYSVLGLEKRHLFSMILKETMIMGFVTLLLGIGVGALFDKLIYAFLQRLIGESTGLVSTF
QVMTIPIVLVIFACIFSFLVLVNGFRLLRLNPLQLTKDGFKGEKKGRFLVIQTFLGLGAMGYGYYLALSVQNP
VIAIMSFFLAVLLVILGTYLLFNAGTTVVLQLLKKKKSYYYKPNNMISISNLVFRMKKNAVGLATIAILSSMVL
VTLVGAASIYAGKKDYLVSAAPHDYSVSGNKVDLTSTKKLMDDFLIKTGEQVNEEVAVSYLFFGIKNQETN
KLTVFTKNERKVVPKSIVLVFSQETFKQLTGKELNLSSNQIALYTKNKTFKTQKSLSIDGKNYQIHRQLGDFI
NKKVPNIYKIIVSDYSYLVVPDIKIFESSMKGTSIAQATYVGVNVKDSTHDAKKNLDLLDQIAGEATKQLAG
QTTGVPESYFSANSRYDAEGMVNGFVGGTFFISIFLSIIFMLGTVLVIYYKQISEGYEDRERFVILQKIGLDDL
QVKQTIRKQVLTIFFLPLIFAFIHLAFAYHMISLIVRIIGVLNPDLMLVVTIIVCGVFFLAYILVFVLTSRSYRRIV
SM
Peptide deformylase, DSM22587
DNA sequence, 615 bps
SEQ ID NO.: 5
ATGGATGCTCAAACCAAAATTATTCGCGCCAGCCACATGATTGATATGAACGATATCATACGCGAAG
GCAACCCAACCTTGCGTGCTGTCGCTGAAGACGTAACCCTACCACTTTCAGATGAAGATATTATCCTT
GGTGAAAAAATGATGCAGTTTCTTCGTAATTCACAGGACCCTGTTATCGCTGAAAAAATGGGACTTC
GAGGAGGTGTTGGTCTTGCAGCACCACAATTAGATATTTCAAAACGCATTATTGCTGTTCTCGTTCCA
AATCCTGAAGACGCTAAGGGGAATCCACCTAAAGAAGCTTATAGCCTTCAAGAAATCATGTATAATC
CTAAAGTAGTTGCTCATTCTGTTCAGGAGGCTGCTCTAGGTAACGGTGAAGGATGCCTTTCAGTCGA
TCGCGACGTTCCTGGATATGTCGTTCGCCATGCTCGTGTTACTATTGAATACTTCAACAAAGAGGGTG
AAAAGAAACGTATTAAACTCCGTGGTTACGACTCAATCGTTGTTCAACATGAAATCGACCATACTAAC
GGTATCATGTTCTACGACCGTATCAATAAAGACAATCCATTTACTATCAAGGATGGACTCTTGATTAT
CGAATAA
AA sequence, 204 amino acids
SEQ ID NO.: 6
MDAQTKIIRASHMIDMNDIIREGNPTLRAVAEDVTLPLSDEDIILGEKMMQFLRNSQDPVIAEKMGLRG
GVGLAAPQLDISKRIIAVLVPNPEDAKGNPPKEAYSLQEIMYNPKVVAHSVQEAALGNGEGCLSVDRDVP
GYVVRHARVTIEYFNKEGEKKRIKLRGYDSIVVQHEIDHTNGIMFYDRINKDNPFTIKDGLLIIE
Peptide deformylase, DSM22933
DNA sequence, 615 bps
SEQ ID NO.: 7
ATGGATGCTCAAACCAAAATTATTCGCGCCAGCCACATGATTGATATGAACGATATCATACGCGAAG
GCAACCCAACCTTGCGTGCTGTCGCTGAAGACGTAACCCTACCACTTTCAGATGAAGATATTATCCTT
GGTGAAAAAATGATGCAGTTTCTTCGTAATTCACAGGACCCTGTTATCGCTGAAAAAATGGGACTTC
GAGGAGGTGTTGGTCTTGCAGCACCACAATTAGATATTTCAAAACGCATTATTGCTGTTCTCGTTCCA
AATCCTGAAGACGCTAAGGGGAATCCACCTAAAGAAGCTTATAGCCTTCAAGAAATCATGTATAATC
CTAAAGTAGTTGCTCATTCTGTTCAGGAGGCTGCTCTAGGTAACGGTGAAGGATGCCTTTCAGTCGA
TCGCGACGTTCCTGGATATGTCGTTTGCCATGCTCGTGTTACTATTGAATACTTCAACAAAGAGGGTG
AAAAGAAACGTATTAAACTCCGTGGTTACGACTCAATCGTTGTTCAACATGAAATCGACCATACTAAC
GGTATCATGTTCTACGACCGTATCAATAAAGACAATCCATTTACTATCAAGGATGGACTCTTGATTAT
CGAATAA
AA sequence, 204 amino acids
SEQ ID NO.: 8
MDAQTKIIRASHMIDMNDIIREGNPTLRAVAEDVTLPLSDEDIILGEKMMQFLRNSQDPVIAEKMGLRG
GVGLAAPQLDISKRIIAVLVPNPEDAKGNPPKEAYSLQEIMYNPKVVAHSVQEAALGNGEGCLSVDRDVP
GYVVCHARVTIEYFNKEGEKKRIKLRGYDSIVVQHEIDHTNGIMFYDRINKDNPFTIKDGLLIIE
Branched-chain amino acid transport ATP-binding protein LivG, DSM22587
DNA sequence, 765 bps
SEQ ID NO.: 9
ATGGCACTTCTTGAAGTTAAAAATTTAACTAAAAACTTTGGTGGTTTGACTGCTGTTGGTGATGTTTC
AATGGAACTCAATGAAGGTGAGTTGGTTGGGCTAATAGGGCCAAACGGTGCTGGTAAAACAACCTT
GTTCAACCTTTTGACTGGTGTCTATGAGCCAAGTGAAGGGACTGTAACGCTTGATGGTATAGTTCTCA
ACGGTAAAGCACCTTACAAGATTGCGTCACTCGGTTTGTCACGTACTTTCCAAAATATCCGCCTTTTCA
AAGACATGACTGTACTTGAAAATGTTCTTGTTGGTTTATCAAATAAGCAACCTTCAAATTTCTTTGCAT
CTCTTTTGCGCTTGCCTAAGTACTATTCAAGTGAGGAAGAGTTGAAAGACAAAGCTATGAAGCTCTT
GGCTATCTTTAACTTGGATGGTGAGGCAGATACGCTTGCGAAAAACTTGGCTTATGGACAACAACGT
CACTTGGAGATTGTTCGTGCGCTTGCAACGGAACCTAAAATTCTTTTCCTCGATGAACCAGCTGCTGG
TATGAACCCACAAGAAACAGCTGAGTTGACTGCTCGTATTCGTCAAATTCAAAAAGATTTCGGTATTA
CAATTATCTTGATTGAGCACGATATGAGTTTGGTCATGGATGTCACTGAGCGTATCTATGTTTTAGAA
TATGGACGCTTGATTGCAGAAGGAACCCCTGATGAAATTAAGAATAACAAGCGTGTTATCGAAGCTT
ACTTGGGAGGTGAAGCATAA
AA sequence, 254 amino acids
SEQ ID NO.: 10
MALLEVKNLTKNFGGLTAVGDVSMELNEGELVGLIGPNGAGKTTLFNLLTGVYEPSEGTVTLDGIVINGK
APYKIASLGLSRTFQNIRLFKDMTVLENVLVGLSNKQPSNFFASLLRLPKYYSSEEELKDKAMKLLAIFNLDG
EADTLAKNLAYGQQRHLEIVRALATEPKILFLDEPAAGMNPQETAELTARIRQIQKDFGITIILIEHDMSLV
MDVTERIYVLEYGRLIAEGTPDEIKNNKRVIEAYLGGEA
Branched-chain amino acid transport ATP-binding protein LivG, DSM22933
DNA sequence, 765 bps
SEQ ID NO.: 11
ATGGCACTTCTTGAAGTTAAAAATTTAACTAAAAACTTTGGTGGTTTGACTGCTGTTGGTGATGTTTC
AATGGAACTCAATGAAGGTGAGTTGGTTGGGCTAATAGGGCCAAACGGTGCTGGTAAAACAACCTT
GTTCAACCTTTTGACTGGTGTCTATGAGCCAAGTGAAGGGACTGTAACGCTTGATGGTATAGTTCTCA
ACGGTAAAGCACCTTACAAGATTGCGTCACTCGGTTTGTCACGTACTTTCCAAAATATCCGCCTTTTCA
AAGACATGACTGTACTTGAAAATGTTCTTGTTGGTTTATCAAATAAGCAACCTTCAAATTTCTTTGCAT
CTCTTTTGCGCTTGCCTAAGTACTATTCAAGTGAGGAAGAGTTGAAAGACAAAGCTATGAAGCTCTT
GGCTATCTTTAACTTGGATGGTGAGGCAGATACGCTTGCGAAAAACTTGGCTTATGGACAACAACGT
CACTTGGAGATTGTTCGTGCGCTTGCAACGGTACCTAAAATTCTTTTCCTCGATGAACCAGCTGCTGG
TATGAACCCACAAGAAACAGCTGAGTTGACTGCTCGTATTCGTCAAATTCAAAAAGATTTCGGTATTA
CAATTATCTTGATTGAGCACGATATGAGTTTGGTCATGGATGTCACTGAGCGTATCTATGTTTTAGAA
TATGGACGCTTGATTGCAGAAGGAACCCCTGATGAAATTAAGAATAACAAGCGTGTTATCGAAGCTT
ACTTGGGAGGTGAAGCATAA
AA sequence, 254 amino acids
SEQ ID NO.: 12
MALLEVKNLTKNFGGLTAVGDVSMELNEGELVGLIGPNGAGKTTLFNLLTGVYEPSEGTVTLDGIVINGK
APYKIASLGLSRTFQNIRLFKDMTVLENVLVGLSNKQPSNFFASLLRLPKYYSSEEELKDKAMKLLAIFNLDG
EADTLAKNLAYGQQRHLEIVRALATVPKILFLDEPAAGMNPQETAELTARIRQIQKDFGITIILIEHDMSLV
MDVTERIYVLEYGRLIAEGTPDEIKNNKRVIEAYLGGEA

### DETAILED DESCRIPTION

### Definitions

All definitions of herein relevant terms are in accordance of what would be understood by the skilled person in relation to the herein relevant technical context.

In the context of the present application, the term “milk” is used in its common meaning to refer to liquids produced by the mammary glands of animals (e.g., cows, sheep, goats, buffaloes, camel, etc.).

The term “milk substrate” may be any raw and/or processed milk material that can be subjected to fermentation according to the method of the invention. Thus, useful milk substrates include, but are not limited to, solutions/suspensions of any milk, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g., being substantially pure mammalian milk, or reconstituted milk powder. Preferably, at least part of the protein in the milk substrate is proteins naturally occurring in mammalian milk, such as casein or whey protein. However, part of the protein may be proteins which are not naturally occurring in milk.

Prior to fermentation, the milk substrate may be homogenized and pasteurized according to methods known in the art.

“Homogenizing” as used in the context of the present invention in any of its embodiments, means intensive mixing to obtain a soluble suspension or emulsion. If homogenization is performed prior to fermentation, it may be performed to break up the milk fat into smaller sizes so that it no longer separates from the milk. This may be accomplished by forcing the milk at high pressure through small orifices.

“Pasteurizing” as used in the context of the present invention in any of its embodiments, means treatment of the milk base to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the context of the present invention in any of its embodiments means the conversion of carbohydrates into acids or alcohols or a mixture of both—through the action of microorganisms (e.g., lactic acid bacteria (LAB)). Fermentation processes to be used in production of food products such as dairy products are well known and the person of skill in the art will know how to select suitable process conditions, such as temperature, oxygen, amount of microorganism(s) and process time. Fermentation conditions are selected so as to support the achievement of the present invention, e.g., to obtain a fermented food product such as a fermented milk product, like a dairy product, preferably a fermented food product which has improved texture as compared to a food product produced with a method which does not involve the use of the strains of the present invention or the use of the composition of the present invention, in any of its embodiments.

The terms “fermented milk” and “dairy” are used interchangeably herein. In the context of the present invention in any of its embodiments, the expression “fermented milk product” means a food or feed product wherein the preparation of the food or feed product involves fermentation of a milk base with a lactic acid bacterium. “Fermented milk product” as used herein includes but is not limited to products such as thermophilic fermented milk products or mesophilic fermented milk products. The term “thermophilic fermentation” herein refers to fermentation at a temperature above about 35° C., such as between about 35° C. to about 45° C. The term “mesophilic fermentation” herein refers to fermentation at a temperature between about 22° C. and about 35° C.

In the context of the present invention in any of its embodiments, the expression “lactic acid bacteria” (“LAB”) designates food-grade bacteria producing lactic acid as the major metabolic end-product of carbohydrate fermentation. These bacteria are related by their common metabolic and physiological characteristics and are Gram positive, low-GC, acid tolerant, non-sporulating, rod-shaped bacilli or cocci. During the fermentation stage, the consumption of carbohydrate by these bacteria causes the formation of lactic acid, reducing the pH and leading to the formation of a protein coagulum. These bacteria are thus responsible for the acidification of milk and for the texture of the dairy product. The S. thermophilus strains of the present invention are classified as lactic acid bacteria.

In the present context the term “starter culture” is a culture which is a preparation of one or 30 more bacterial strains (such as lactic acid bacteria strains) to assist the fermentation process in preparation of fermented products such as various foods, feeds and beverages.

In the present context, a “yoghurt starter culture” is a bacterial culture which comprises at least one Lactobacillus delbrueckii subsp bulgaricus (L. bulgaricus) strain and at least one Streptococcus thermophilus (S. thermophilus) strain. In accordance herewith, a “yoghurt” refers to a fermented milk product obtainable by inoculating and fermenting a milk substrate with a composition comprising a L. bulgaricus strain and a S. thermophilus strain.

“Texture” is also an important quality factor for fermented milk products and consumer acceptance is often closely linked to texture properties. The texture of fermented milk is dependent on the exocellular polysaccharide structures, the bacteria used for fermentation and process parameters as well as milk composition. In the context of the present invention, the rheological properties (texture) of a fermented milk product, such as viscosity, can be measured as a function of shear stress of the fermented milk product, as described below. Viscosity can also be measured by the viscosity pipette test as described in Examples 2 and 3 below. In addition, texturizing properties can also be evaluated with the aspiration test as described in Example 4 below.

A “texturing strain” in the present specification and claims is a strain which preferably generates fermented milks having under the conditions described below and as exemplified in the examples herein, a shear stress preferably greater than 40 Pa, such as 44 Pa or higher, measured at shear rate 300 s⁻¹. A strain of S. thermophilus can be defined as strongly texturing in that it generates fermented milks having, under the same conditions, a shear stress of 53 Pa or higher, measured at shear rate 300 s⁻¹.

200 ml milk (3.6% protein, 1.5% or 3% fat) is heated to 95° C. for 5 min, followed by cooling to inoculation temperature (43° C.), and inoculated with 0.02% of FD-DVS starter culture comprising the lactic acid bacterium strain, and left at inoculation temperature (43° C.) until pH 4.60, followed by storage at 6° C. for 7 days, followed by gently stirring and measuring the shear stress at shear rate 300 s⁻¹ at 13° C.

In connection with the present invention, “shear stress” may be measured by the following method: When the pH of the fermented milk (e.g., mammalian- or plant-based milk) reached pH^{˜}4.60 at the incubation temperature, e.g., 43° C., the fermented milk product was cooled down by transferring the container to ice water and optionally stored at 6° C. for 7 days. The fermented milk sample was manually stirred gently by means of a stick fitted with a perforated disc until homogeneity of the sample. The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:
Holding time (to rebuild to somewhat original structure)
5 minutes without any physical stress (oscillation or rotation) applied to the sample.
Oscillation step (to measure the elastic and viscous modulus, G′ and G″, respectively, therefore calculating the complex modulus G*)
   Constant strain=0.3%, frequency (f)=[0.5 . . . 8] Hz
   6 measuring points over 60 s (one every 10 s)
Rotation step (to measure shear stress at 300 1/s)
Two steps were designed:
Shear rate=[0.3−300] 1/s and 2) Shear rate=[275−0.3] 1/s.

Each step contained 21 measuring points over 210 s (on every 10 s). The shear stress at 300 1/s (300 s⁻¹) was chosen for further analysis, as this correlates to mouth thickness when swallowing a fermented milk product.

As used herein, the “viscosity pipette test” refers to a method of determining the viscosity of a product by determining the efflux time from a volumetric pipette. A longer efflux time corresponds to higher viscosity, see also Example 2:

Coagulated milk was made from 200 mL skimmed milk inoculated with 1% of the bacterial strain(s) to be tested (from an overnight culture grown in skimmed milk at 37° C.), and incubated for 20 h at 42° C. or at 37° C. The viscosity of the coagulated milk was measured with a 25 mL volumetric pipette where the efflux time of said coagulated milk from the pipette was measured in triplicates. The coagulated milk is stirred carefully with a spoon to homogenize. The 25 mL volumetric pipette is then filled and the time to empty the pipette by gravity force is measured. The time it takes to empty 25 mL of coagulated milk from the pipette is noted as seconds.

As used herein, the term “bacteriophage” has its conventional meaning as understood in the art, i.e., a virus that selectively infects one or more bacteria. Many bacteriophages are specific to a particular genus or species or strain of bacteria. The term “bacteriophage” is synonymous with the term “phage”. Bacteriophages may include, but are not limited to, bacteriophages that belong to any of the following virus families: Corticoviridae, Cystoviridae, Inoviridae, Leviviridae, Microviridae, Myoviridae, Podoviridae, Siphoviridae, or Tectiviridae. The bacteriophage may be a lytic bacteriophage or a lysogenic bacteriophage. A lytic bacteriophage is one that follows the lytic pathway through completion of the lytic cycle, rather than entering the lysogenic pathway. A lytic bacteriophage undergoes viral replication leading to lysis of the cell membrane, destruction of the cell, and release of progeny bacteriophage particles capable of infecting other cells. A lysogenic bacteriophage is one capable of entering the lysogenic pathway, in which the bacteriophage becomes a dormant, passive part of the cell's genome through prior to completion of its lytic cycle.

In the context of the present invention, a “phage resistant mutant” refers to a bacterial strain which has developed mechanisms to defend against phages. In one embodiment, the lactic acid bacterium according to the present invention is resistant to one or more bacteriophage or one or more sets of bacteriophages; in another embodiment, the lactic acid bacterium according to the present invention is resistant to the same bacteriophage that a strain deposited according to the present invention is resistant to. Preferably, the lactic acid bacterium according to the present invention is resistant to phage DSM24022. In the present context, the term “phage robust” is used interchangeable with the term “phage resistant”.

Phage resistant mutants according to the present invention can be obtained as described in Example 1 below.

In the present context, the terms “strains derived from”, “derived strain” or “mutant” should be understood as a strain derived from another strain (or the “mother strain”) by means of, e.g., genetic engineering, radiation and/or chemical treatment, and/or selection, adaptation, screening, etc. The mutant can also be a spontaneously occurring mutant. It is preferred that the derived strain is a functionally equivalent mutant, e.g., a strain that has substantially the same, or improved, properties with respect to, e.g., growth and acidification properties as the mother strain. Such a derived strain is a part of the present invention. Especially, the term “derived strain” or “mutant” refers to a strain obtained by subjecting a mother strain to any conventionally used mutagenizing treatment including treatment with a chemical mutagen such as ethane methane sulphonate (EMS) or N-methyl-N′-nitro-N-nitroguanidine (NTG), UV light or to a spontaneously occurring mutant. Mutants can also be generated by site directed mutagenesis.

A mutant may have been subjected to several mutagenizing treatments (a single treatment should be understood as one mutagenizing step followed by a screening/selection step), but it is presently preferred that no more than 20, no more than 10, or no more than 5, treatments are carried out. In a presently preferred derived strain, less than 1%, or less than 0.1%, less than 0.01%, less than 0.001% or even less than 0.0001% of the nucleotides in the bacterial genome have been changed (such as by replacement, insertion, deletion or a combination thereof) compared to the mother strain.

In the present context, the term “variant” should be understood as a strain which is functionally equivalent to a strain of the invention, e.g. having substantially the same, or improved, properties e.g. regarding viscosity, gel stiffness, mouth coating, flavor, post acidification, acidification speed, and/or phage robustness). Such variants, which may be identified using appropriate screening techniques, are a part of the present invention.

The term “sequence identity” relates to the relatedness between two nucleotide sequences or between two amino acid sequences. Algorithms for aligning sequences and determining the degree of sequence identity between them are well known in the art. For purposes of the present invention, the degree of sequence identity between two nucleotide sequences or two amino acid sequences is determined, for example, using multiple sequence alignment tool Clustal Omega (https://www.ebi.ac.uk/Tools/msa/clustalo/; Sievers et al., 2011) with standard parameters.

For purposes of the present invention, the degree of identity between two amino acid sequences is determined, for example, using the Needleman-Wunsch algorithm (Needleman and Wunsch (1970) J. Mol. 25 Biol. 48: 443-453) as implemented in the Needle program of the EMBOSS package (EMBOSS: The European Molecular Biology Open Software Suite, Rice et a. (2000) Trends in Genetics 16:276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labelled “longest identity” (obtained using the -no brief 30 option) is used as the percent identity and is calculated as follows:

$$( Identical Residues \times 100 ) / ( Length of Alignment - Total Number of Gaps in Alignment ) .$$

For the purpose of the present invention a process may be carried out for aligning nucleotide sequences using blastn as provided by the National Center for Biotechnology Information (NCBI) on https://blast.ncbi.nlm.nih.gov applying standard parameters.

In the present description and claims the conventional one-letter and three-letter codes for amino acid residues are used. For ease of reference, amino acid changes in mutants and variants of the invention are described by use of the following nomenclature: amino acid residue in the parent protein; position; substituted amino acid residue(s). According to this nomenclature, the substitution of, for instance, a glutamic acid residue for a valine residue at position 169 is indicated as Glu169Val or E169V.

In the context of the present invention, a mutation in the gene (“gene mutation”) is to be understood as an alteration in the nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift. In the context of the present invention, a “deletion” is to be understood as a genetic mutation resulting in the removal of one or more nucleotides of a nucleotide sequence of the genome of an organism; a “insertion” is to be understood as the addition of one or more nucleotides to the nucleotide sequence; a “substitution” (or “point mutation”) is to be understood as a genetic mutation where a nucleotide of a nucleotide sequence is substituted by another nucleotide. In the context of the present invention, a mutation in a gene refers to an alteration in the nucleotide sequence of any of the elements comprised in a gene. For instance, a gene may comprise several elements or parts such as different regulatory sequences (enhancers, silencers, promoters, 5′ and 3′UTRs, etc.) and open reading frame regions (which comprise introns and exons). Hence, in the context of the present invention, a mutation in a certain gene is to be understood as an alteration in the nucleotide sequence of said gene, that alteration being either in a regulatory element of the gene (such as in the promoter of the gene) and/or in the open reading frame of the gene (such as in an exon). If the mutation occurs, e.g., in the sequence of an exon, the mutation may lead to a different amino acid in the translated protein. If the mutation occurs, e.g., in a regulatory region of the gene, the mutation may lead to an enhanced (or decreased) expression of the gene, e.g., to an enhanced (or decreased) amount of protein.

In the context of the present invention, a substitution or a mutation of one amino acid or nucleotide to another amino acid or nucleotide at a position corresponding to a certain position in a certain sequence means that, in the mutated amino acid or nucleotide sequence, there may be further mutations (e.g., deletions, insertions, substitutions, etc.) besides the specific substitution or mutation at the specified position (or at a position corresponding to that specific position, in case, e.g., where there are deletions or insertions in the mutated sequence). Hence, in the context of the present invention, for instance, the phrase “the mutation in the branched chain amino acid transport ATP-binding protein LivG (TC 3.A.1.4.1) is a substitution of Glutamic acid to Valine at a position corresponding to position 169 of SEQ ID NO.: 12 and/or a mutation of nucleotide A to nucleotide T at a position corresponding to position 506 of SEQ ID NO.: 11” can mean:
(i) that, in the mutated amino acid or nucleotide sequence of the branched chain amino acid transport ATP-binding protein LivG (TC 3.A.1.4.1), there is a substitution of Glutamic acid to Valine exactly at position 169 of SEQ ID NO.: 12 or a substitution (mutation) of nucleotide A to nucleotide T exactly at position 506 of SEQ ID NO.: 11. Of course, further substitutions or mutations of SEQ ID NO.: 12 or in SEQ ID NO.: 11 are not excluded;
(ii) that, in the case that there are further mutations in the sequence such as insertions or deletions, the specific substitution of Glutamic acid to Valine or of nucleotide A to nucleotide T may no longer be exactly at position 169 of SEQ ID NO.: 12 or exactly at position 506 of SEQ ID NO.: 11, because of the further mutations such as insertions or deletions. In these cases, the recited substitution of Glutamic acid to Valine or of nucleotide A to nucleotide T should be at a position which, taking into account the further mutations in the sequence, would correspond to position 169 of SEQ ID NO.: 12 or to position 506 in SEQ ID NO.: 11. The skilled person is able to identify a “position corresponding to a certain position in a certain sequence”, e.g., by aligning the sequences and finding the position which would correspond to a certain position in the original sequence.

A corresponding meaning would apply to the phrases “the mutation in the ABC transporter permease protein is a substitution of Leucine to Phenylalanine at a position corresponding to position 190 of SEQ ID NO.: 4 and/or a mutation of nucleotide C to nucleotide T at a position corresponding to position 568 of SEQ ID NO.: 3” and “the mutation in the peptide deformylase protein is a substitution of Arginine to Cysteine at a position corresponding to position 144 of SEQ ID NO.: 8 and/or a mutation of nucleotide C to nucleotide T at a position corresponding to position 430 of SEQ ID NO.: 7”.

In the present description and claims the conventional one-letter code for nucleotides is used following the analogous principles as described for amino acids nomenclature supra.

As used herein, the term “about” (or “around”) means the indicated value ±1% of its value, or the term “about” means the indicated value ±2% of its value, or the term “about” means the indicated value ±5% of its value, the term “about” means the indicated value ±10% of its value, or the term “about” means the indicated value ±20% of its value, or the term “about” means the indicated value ±30% of its value; preferably the term “about” means exactly the indicated value (±0%).

Throughout the description and claims the word “comprise” and variations of the word (e.g., “comprising”, “having”, “including”, “containing”) typically is not limiting and thus does not exclude other features, which may be for example technical features, additives, components, or steps. However, whenever the word “comprise” is used herein, this also includes a special embodiment in which this word is understood as limiting; in this particular embodiment the word “comprise” has the meaning of the term “consist of”.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

According to the present invention, viscosity can be (and is preferably) measured as described in Examples 2 and 3. According to the present invention, aspiration can be (and is preferably) measured as described in Example 4. According to the present invention, rheological properties of a lactic acid bacterium or a blend such as shear stress can be (and are preferably) measured as described in Example 5.

Streptococcus thermophilus Strains

The inventors have surprisingly identified S. thermophilus strains that fulfil the needs of the industry. The new strains show, e.g., improved rheological properties (e.g., texture), when applied alone or as part of a mixed culture in a dairy substrate when compared to its mother strain. The novel S. thermophilus strains have the capacity to be used in, e.g., dairy cultures such as yoghurt cultures to obtain improved rheological parameters, such as shear stress, viscosity and gel firmness of the final product. Rheology is closely linked to sensory quality of the product and the interplay between rheology and taste in the final product is therefore of outmost importance.

The inventors have surprisingly found that S. thermophilus strains having a mutation in the branched chain amino acid transport ATP-binding protein LivG (TC 3.A.1.4.1) gene have better texturing properties than their mother strain, i.e., than a strain which does not have the same mutation in the branched chain amino acid transport ATP-binding protein LivG (TC 3.A.1.4.1) gene. In particular, the present inventors have found that a mutation in the branched chain amino acid transport ATP-binding protein LivG (TC 3.A.1.4.1) gene, wherein the mutation is a substitution of Glutamic acid to Valine at a position corresponding to position 169 of SEQ ID NO.: 12 is linked to better texturing properties. For instance, the present inventors have surprisingly found that S. thermophilus strains having a mutation in the branched chain amino acid transport ATP-binding protein LivG (TC 3.A.1.4.1) gene, wherein the mutation is a substitution of nucleotide A to nucleotide T at a position corresponding to position 506 of SEQ ID NO.: 11 show better texturing properties than its mother strain. This is shown in the examples. Hence, the present inventors have surprisingly found that S. thermophilus strains having a Valine at a position corresponding to position 169 of SEQ ID NO.: 12 and/or a T at a position corresponding to position 506 of SEQ ID NO.: 11 (the branched chain amino acid transport ATP-binding protein LivG (TC 3.A.1.4.1) gene) show better texturing properties than S. thermophilus strains which do not have a Valine at a position corresponding to position 169 of SEQ ID NO.: 12 (e.g., strains which may have a Glutamic acid at a position corresponding to position 169 of SEQ ID NO.: 12) and/or a T at a position corresponding to position 506 of SEQ ID NO.: 11 (e.g., strains which may have an A at a position corresponding to position 506 of SEQ ID NO.: 11).

Hence, a first aspect of the present invention relates to a S. thermophilus strain having (i) a mutation in the branched chain amino acid transport ATP-binding protein LivG (TC 3.A.1.4.1) gene, wherein the mutation is preferably a substitution of Glutamic acid to Valine at a position corresponding to position 169 of SEQ ID NO.: 12. In another aspect, the present invention relates to a S. thermophilus strain having (i) a mutation in the branched chain amino acid transport ATP-binding protein LivG (TC 3.A.1.4.1) gene, wherein the mutation is preferably a substitution of nucleotide A to nucleotide T at a position corresponding to position 506 of SEQ ID NO.: 11. Hence, the present invention further relates to a S. thermophilus strain having a Valine at a position corresponding to position 169 of SEQ ID NO.: 12 and/or a T at a position corresponding to position 506 of SEQ ID NO.: 11 (the branched chain amino acid transport ATP-binding protein LivG (TC 3.A.1.4.1) gene).

Further the inventors have surprisingly found that S. thermophilus strains having a mutation in the ABC transporter permease protein gene have better texturing properties than their mother strain, i.e., than a strain which does not have the same mutation in the ABC transporter permease protein gene. In particular, the present inventors have found that a mutation in the ABC transporter permease protein gene, wherein the mutation is a substitution of Leucine to Phenylalanine at a position corresponding to position 190 of SEQ ID NO.: 4, is linked to better texturing properties. For instance, the present inventors have surprisingly found that S. thermophilus strains having a mutation in the ABC transporter permease protein gene, wherein the mutation is a substitution of nucleotide C to nucleotide Tat a position corresponding to position 568 of SEQ ID NO.: 3 show better texturing properties than the mother strain. This is shown in the examples. Hence, the present inventors have surprisingly found that S. thermophilus strains having a Phenylalanine at a position corresponding to position 190 of SEQ ID NO.: 4 and/or a T at a position corresponding to position 568 of SEQ ID NO.: 3 (ABC transporter permease protein gene) show better texturing properties than S. thermophilus strains which do not have a Phenylalanine at a position corresponding to position 190 of SEQ ID NO.: 4 (e.g., strains which have a Leucine at a position corresponding to position 190 of SEQ ID NO.: 4) and/or strains which do not have a T at a position corresponding to position 568 of SEQ ID NO.: 3 (e.g., strains which have a C at a position corresponding to position 568 of SEQ ID NO.: 3).

Hence, the S. thermophilus strain of the invention has (ii) a mutation in the ABC transporter permease protein gene, wherein the mutation is preferably a substitution of Leucine to Phenylalanine at a position corresponding to position 190 of SEQ ID NO.: 4. For instance, the S. thermophilus strain of the invention has (ii) a mutation in the ABC transporter permease protein gene, wherein the mutation is preferably a substitution of nucleotide C to nucleotide T at a position corresponding to position 568 of SEQ ID NO.: 3. Hence, the present invention further relates to a S. thermophilus strain having (ii) a phenylalanine at a position corresponding to position 190 of SEQ ID NO.: 4 and/or a T at a position corresponding to position 568 in SEQ ID NO.: 3 (ABC transporter permease protein gene).

Further the inventors have surprisingly found that S. thermophilus strains having a mutation in the peptide deformylase protein gene have better texturing properties than their mother strain, i.e., than a strain which does not have the same mutation in the peptide deformylase protein gene. In particular, the present inventors have found that a mutation in the peptide deformylase protein gene, wherein the mutation is a substitution of Arginine to Cysteine at a position corresponding to position 144 of SEQ ID NO.: 8 is linked to better texturing properties. For instance, the present inventors have surprisingly found that S. thermophilus strains having a mutation in the peptide deformylase protein gene, wherein the mutation is a substitution of nucleotide C to nucleotide T at a position corresponding to position 430 of SEQ ID NO.: 7 show better texturing properties than the mother strain. This is shown in the examples. Hence, the present inventors have surprisingly found that S. thermophilus strains having a cysteine at a position corresponding to position 144 of SEQ ID NO.: 8 and/or a T at a position corresponding to position 430 of SEQ ID NO.: 7 (peptide deformylase protein gene) show better texturing properties than S. thermophilus strains which do not have a cysteine at a position corresponding to position 144 of SEQ ID NO.: 8 (e.g., strains which have an Arginine at a position corresponding to position 144 of SEQ ID NO.: 8) and/or strains which do not have a T at a position corresponding to position 430 of SEQ ID NO.: 7 (e.g., strains which have a C at a position corresponding to position 430 of SEQ ID NO.: 7).

Hence, the S. thermophilus strain of the invention has (iii) a mutation in the peptide deformylase protein gene, wherein the mutation is preferably a substitution of Arginine to Cysteine at s position corresponding to position 144 of SEQ ID NO.: 8. For instance, the S. thermophilus strain of the invention has (iii) a mutation in the peptide deformylase protein gene, wherein the mutation is preferably a substitution of nucleotide C to nucleotide T at a position corresponding to position 430 of SEQ ID NO.: 7. Hence, the present invention relates to a S. thermophilus strain having (iii) a cysteine at a position corresponding to position 144 of SEQ ID NO.: 8 and/or a T at a position corresponding to position 430 of SEQ ID NO.: 7 (peptide deformylase protein gene).

Preferably, the S. thermophilus strain of the invention has at least two of the mutations (i)-(iii) as described above, preferably mutations (i) and (ii), i.e., (i) a mutation in the branched chain amino acid transport ATP-binding protein LivG (TC 3.A.1.4.1) gene, wherein the mutation is preferably a substitution of Glutamic acid to Valine at a position corresponding to position 169 of SEQ ID NO.: 12 or wherein the mutation is preferably a substitution of nucleotide A to nucleotide T at a position corresponding to position 506 of SEQ ID NO.: 11 and (ii) a mutation in the ABC transporter permease protein gene, wherein the mutation is preferably a substitution of Leucine to Phenylalanine at a position corresponding to position 190 of SEQ ID NO.: 4 or wherein the mutation is preferably a substitution of nucleotide C to nucleotide T at a position corresponding to position 568 of SEQ ID NO.: 3. In another aspect, the S. thermophilus strain of the invention has mutations (i) and (iii) as described above, or has mutations (ii) and (iii) as described above.

In one embodiment, the S. thermophilus strain of the invention has all three mutations in the protein sequences and/or in the nucleotide sequences as described above (i) to (iii).

In one embodiment, the branched chain amino acid transport ATP-binding protein LivG (TC 3.A.1.4.1) gene of the S. thermophilus strain of the invention has a T at a position corresponding to position 506 of SEQ ID NO.: 11, and/or the branched chain amino acid transport ATP-binding protein LivG (TC 3.A.1.4.1) has a valine at a position corresponding to position 169 of SEQ ID NO.: 12. In a further preferred aspect, the ABC transporter permease protein gene of the S. thermophilus strain of the invention has a T at a position corresponding to position 568 of SEQ ID NO.: 3 and/or the ABC transporter permease protein has a phenylalanine at a position corresponding to position 190 of SEQ ID NO.: 4. In another aspect, the S. thermophilus strain of the invention has both (i) and (ii) as described above. In another aspect, the S. thermophilus strain of the invention has (i) and (iii) as described above. In a further aspect, the S. thermophilus strain of the invention has (ii) and (iii) as described above.

In a preferred aspect, the S. thermophilus strain of the invention has (i), (ii) and (iii) as described above.

Preferably, the S. thermophilus strain of the invention generates higher shear stress and/or higher efflux time in a viscosity pipette test (measured as described herein and in the Examples) than a strain not comprising any of the mutations (i), (ii), or (iii) as described above, when used for fermenting milk. In one embodiment of the invention the strain not comprising any of the mutations (i), (ii), or (iii) as described above is the mother strain from which the S. thermophilus strain of the invention is derived. In one embodiment of the invention the strain not comprising any of the mutations (i), (ii), or (iii) as described above is the strain DSM22587.

In one embodiment of the invention, the mutant strain is phage resistant, substantial phage resistant, and/or possesses increased phage resistance compared to its mother stain. This means that the mother strain is susceptible to be infected (lysed) by a phage, but the mutant strain is resistant to the infection (lysis) by that same phage. Preferably, the S. thermophilus strain of the invention is resistant to phage DSM24022. In one embodiment, the S. thermophilus strain of the invention is a phage resistant mutant from strain DSM22587. Preferably, the S. thermophilus strain of the invention is a mutant from strain DSM22587, which mutant is resistant to phage DSM24022.

Even more preferably, the S. thermophilus strain of the invention is DSM22933 or a mutant or variant thereof. Preferably, the mutant or variant shows the same or similar texturing properties as DSM22933, e.g., the same or similar shear stress and/or the same or similar viscosity properties. Hence, it is contemplated that the S. thermophilus strains of the invention, including the mutants and variants of DSM22933, show at least the same or similar shear stress and/or viscosity characteristics and/or texturizing properties, as defined in the present examples, as DSM22933. Hence, the present invention further provides the S. thermophilus strain DSM22933 or a mutant or variant thereof.

In the present context the term “same or similar shear stress” is to be understood as a range spanning from 10% below the shear stress characteristics of DSM22933 to 10% above the shear stress characteristics of DSM22933, the range may also be 9% below/above the shear stress characteristics of DSM22933, such as 8% below/above of the shear stress characteristics of DSM22933, e.g. 7% below/above of the shear stress characteristics of DSM22933, such as 6% below/above of the shear stress characteristics of DSM22933, e.g. 5% below/above of the shear stress characteristics of DSM22933, such as 4% below/above of the shear stress characteristics of DSM22933, e.g. 3% below/above of the shear stress characteristics of DSM22933, such as 2% below/above of the shear stress characteristics of DSM22933 or 1% below/above of the shear stress characteristics of DSM22933. The shear stress characteristics of DSM22933 are measured as described in the description, or as described in Example 5. For instance, the shear stress characteristics of DSM2293 can be measured in mixed cultures, as described in Example 5. For instance, the shear stress characteristics of DSM2293 can be measured as follows:

Shear stress data were obtained by inoculating the same microbial cultures in milk (3.6% protein and 1.5% or 3% fat); milk was heated at 95° C. for 5 min and cooled down to the inoculation temperature (43° C.), prior to inoculation with 30% of the texturing strain to be tested, 60% of another S. thermophilus strain and 10% of a Lb. bulgaricus strain, or milk was heated at 95° C. for 5 min and cooled down to the inoculation temperature (43° C.), prior to inoculation with 50% of the texturing strain to be tested, 25% of another S. thermophilus strain and 25% of a Lb. bulgaricus strain, preferably strain DSM26419. The fermentation took place at 43° C. until pH 4.60, followed by cooling to 6° C. and storage for 7 days at 6° C. After the storage, the fermented milk was stirred gently by means of a stick fitted with a bored disc until homogeneity of the sample. Shear stress of the samples was assessed at 13° C. on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and γ′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ was chosen.

In the present context the term “same or similar viscosity properties” is to be understood as a range spanning from 10% below the viscosity properties of DSM22933 to 10% above the viscosity properties of DSM22933, the range may also be 9% below/above the viscosity properties of DSM22933, such as 8% below/above of the viscosity properties of DSM22933, e.g. 7% below/above of the viscosity properties of DSM22933, such as 6% below/above of the viscosity properties of DSM22933, e.g. 5% below/above of the viscosity properties of DSM22933, such as 4% below/above of the viscosity properties of DSM22933, e.g. 3% below/above of the viscosity properties of DSM22933, such as 2% below/above of the viscosity properties of DSM22933 or 1% below/above of the viscosity properties of DSM22933. The viscosity properties of DSM22933 are measured as described herein and/or in Examples 2-3. For instance, the viscosity properties can be measured with the viscosity pipette test, i.e., determining the efflux time from a volumetric pipette. A longer efflux time corresponds to higher viscosity. For example, the viscosity pipette test can be performed as follows:

Coagulated milk was made from 200 mL skimmed milk inoculated with 1% of the bacterial strain(s) to be tested (from an overnight culture grown in skimmed milk at 37° C.), and incubated for 20 h at 42° C. or at 37° C. The viscosity of the coagulated milk was measured with a 25 mL volumetric pipette where the efflux time of said coagulated milk from the pipette was measured in triplicates. The coagulated milk is stirred carefully with a spoon to homogenize. The 25 mL volumetric pipette is then filled and the time to empty the pipette by gravity force is measured. The time it takes to empty 25 mL of coagulated milk from the pipette is noted as seconds.

The viscosity pipette test can be performed with the strain on its own (single strain, e.g., Example 2) or with a mixed culture of the S. thermophilus and an acidifying Lb. bulgaricus strain, preferably strain DSM19251, and optionally with 1% yeast extract (e.g., Example 3).

In the above “characteristics” is to be understood in the context of the definition part where it's stated how to appropriately measure shear stress or viscosity. Methods for determining the texture of fermented products such as dairy products include measuring the shear stress or viscosity properties of the fermented product and are readily available and known in the art and exemplified herein.

In one embodiment, the Streptococcus thermophilus strains of the present invention have improved texturizing properties as described above, while maintaining the growth properties and acidification properties of its parent (mother) strain.

Compositions Comprising the Streptococcus thermophilus Strains

The present invention also provides compositions and starter cultures comprising the S. thermophilus strains of the invention as described above.

Lactic acid bacteria (LAB), including bacteria of the species S. thermophilus, are normally supplied to the dairy industry either as frozen (F-DVS®) or freeze-dried (FD-DVS®) cultures for bulk starter propagation or as so-called “Direct Vat Set” (DVS®) cultures, intended for direct inoculation into a fermentation vessel or vat for the production of a dairy product, such as a fermented milk product. Such lactic acid bacterial cultures are in general referred to as “starter cultures” or “starters”. Hence, the present invention further provides a starter culture or starter, preferably a yoghurt starter culture, comprising the strains of the present invention as described above. The composition or starter culture of the present invention may be frozen or freeze-dried. In addition, the composition or starter culture of the present invention may be provided in liquid form. Thus, in one embodiment, the composition is in frozen, dried, freeze-dried or liquid form. Preferably, the composition and/or starter culture of the present invention is in frozen, spray-dried, freeze-dried, vacuum-dried, air dried, tray dried or liquid form.

The compositions or starter cultures of the present invention may also additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the compositions or starter cultures according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants. In one embodiment of the invention the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity, e.g., cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i.e. the production of, e. g., lactic acid, acetic acid, formic acid and/or propionic acid, or its metabolite producing activity such as the production of aroma compounds such as acetaldehyde, (α-acetolactate, acetoin, diacetyl and 2,3-butylene glycol (2,3-butanediol)).

In one embodiment the compositions or starter cultures of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In one embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In a further aspect, the compositions or starter cultures of the present invention contains or comprises an ammonium salt (e.g. an ammonium salt of an organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH₃), NH₄OH, NH₄⁺, and the like.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

The composition or the starter culture of the present invention may be a mixture or a kit-of-parts comprising:
i) the Streptococcus thermophilus strain of the invention, preferably strain DSM22933, and
ii) ii) a strain belonging to the species Lactobacillus delbrueckii subsp bulgaricus.

In order to obtain the best combination of acidity, taste, texture of a product such as a dairy product, like yoghurt, a combination of S. thermophilus and Lactobacillus delbrueckii subsp bulgaricus is often applied.

In one embodiment, the mixture or kit-of-parts may comprise the S. thermophilus strain of the present invention in combination with one or more Lactobacillus delbrueckii subsp bulgaricus strain(s) and, optionally, one or more S. thermophilus strain(s).

For instance, the mixture or kit-of-parts may comprise the S. thermophilus strain of the present invention in combination with Lactobacillus delbrueckii subsp bulgaricus strain DSM19251. In one embodiment the mixture or kit-of-parts may comprise S. thermophilus strain DSM22933 in combination with Lactobacillus delbrueckii subsp bulgaricus strain DSM19251.

In addition, the composition of the present invention may further comprise yeast extract. Hence, the composition of the present invention may comprise the Streptococcus thermophilus strain of the invention, a strain belonging to Lactobacillus delbrueckii subsp bulgaricus, preferably as defined above, optionally further Streptococcus thermophilus strain(s), preferably as defined above, and optionally yeast extract.

The expression “mixture” means that the S. thermophilus strain(s) and the Lactobacillus delbrueckii subsp bulgaricus strain(s) are physically mixed together. In an embodiment, the S. thermophilus strain(s) and the Lactobacillus delbrueckii subsp bulgaricus strain(s) are in the same box or in the same pouch.

In contrast, the expression “A kit-of-part” comprising S. thermophilus strain(s) and the L. bulgaricus strain means that the culture of the S. thermophilus strain(s) and the L. bulgaricus strain(s) culture are physically separated but intended to be used together. Thus, the culture of the S. thermophilus strain(s) and the L. bulgaricus strain(s) culture are in different boxes or sachets. In an embodiment, the culture of the S. thermophilus strain(s) and the L. bulgaricus strain(s) are under the same format, i.e., are in a frozen format, in the form of pellets or frozen pellets, a powder form, such as a dried or freeze-dried powder.

In one embodiment of the present invention, the composition comprises from 10⁴ to 10¹² CFU (colony forming units)/g of the S. thermophilus strain(s), such as from 10⁵ to 10¹¹ CFU/g, such as from 10⁶ to 10¹⁰ CFU/g, or such as from 10⁷ to 10⁹ CFU/g of the S. thermophilus strain(s). In one embodiment the composition further comprises from 10⁴ to 10¹² CFU/g of the L. bulgaricus strain(s), such as from 10⁵ to 10¹¹ CFU/g, such as from 10⁶ to 10¹⁰ CFU/g, or such as from 10⁷ to 10⁹ CFU/g of the L. bulgaricus strain(s).

### Method for Producing a Fermented Food Product

The present invention further relates to methods of producing a fermented food product comprising at least one stage in which at least one of the S. thermophilus strain as defined in the first aspect of the present invention and/or the composition or starter culture as defined in the second aspect of the present invention are used. The production of the food product is carried out by methods known to the person skilled in the art.

Depending on the product to be produced, the substrate may be a milk substrate. A milk substrate is particularly preferred when fermented milk products such as yoghurt, buttermilk or kefir is the final product. Hence, in one embodiment, the method comprises fermenting a milk substrate with the strain as defined in the first aspect and/or the composition as described in the second aspect of the present invention, in any of its embodiments.

Hence, the present invention provides a food product comprising the strain and/or the composition according to the invention. Preferably, the food product is a dairy product and the method in any of its embodiments comprises fermenting a milk substrate (also referred to as “milk base” in the context of the present invention) with the at least one S. thermophilus and/or with the composition or starter culture according to the invention.

The food product according to the present invention may advantageously further comprise a “thickener” and/or a “stabilizer”, such as pectin (e.g. HM pectin, LM pectin), gelatin, CMC, Soya Bean Fiber/Soya Bean Polymer, starch, modified starch, carrageenan, alginate, and guar gum.

In one embodiment, the food product is a dairy product, as defined above. In a particular embodiment of the invention, the fermented milk product is selected from the group consisting of yoghurt, Mozarella cheese, kefir, sour cream, cheese, quark. Yoghurt is particularly preferred. In one embodiment of the invention, the fermented milk product contains a further food product selected from the group consisting of fruit beverage, cereal products, fermented cereal products, chemically acidified cereal products, soymilk products, fermented soymilk products and any mixture thereof.

In one embodiment the fermented product further comprises an ingredient selected from the group consisting of a fruit concentrate, a syrup, a probiotic bacterial strain or culture, a coloring agent, a thickening agent, a flavoring agent, a preserving agent and mixtures thereof.

Likewise, an enzyme may be added to the substrate e.g. the milk substrate before, during and/or after the fermenting, the enzyme being selected from the group consisting of an enzyme able to crosslink proteins, transglutaminase, an aspartic protease, chymosin, rennet and combinations thereof. In one embodiment the fermented product may be in the form of a stirred type product, a set type product or a drinkable product.

The fermented milk product typically contains protein in a level of between 1.0% to 12.0% by weight, preferably between 2.0% to 10.0% by weight. In a particular embodiment, sour cream contains protein in a level of between 1.0% to 5.0% by weight, preferably between 2.0% to 4.0% by weight. In a particular embodiment, Quark contains protein in a level of between 4.0% to 12.0% by weight, preferably between 5. % to 10.0% by weight.

Preferably, the food product has a texture (as described in the present invention such as in Examples 2-5) as compared to a food product produced with a comparable method which does not involve the use of at least one of the S. thermophilus strains as described in the present invention and/or the use of the composition or starter culture according to the present invention.

### Fermented Food Product Directly Obtainable by the Method of the Invention

In one embodiment the invention also relates to a fermented food product directly obtained by the method of the present invention, or comprising a Streptococcus thermophilus strain according to the invention, or comprising a composition according to the invention. An aspect of the present invention is therefore also a fermented product comprising the Streptococcus thermophilus strains of the present invention and/or the compositions of the present invention. The fermented product may be preferably a dairy product, such as yoghurt.

Method for Manufacturing Streptococcus thermophilus Strains According to the Present Invention

The present invention provides a method for manufacturing Streptococcus thermophilus strains and/or compositions according to the present invention, wherein the method comprises the following steps:
(i) providing a lactic acid bacterial strain as the mother strain;
(ii) exposing the mother strain to any mutagenizing treatment including treatment with a chemical mutagen or UV light and/or performing site directed mutagenesis as defined herein on to the mother strain;
(iii) screening for a mutant strain comprising at least one, preferably two and, more preferably all three of the following mutations:
   a) a mutation in the branched chain amino acid transport ATP-binding protein LivG (TC 3.A.1.4.1) gene;
   b) a mutation in the ABC transporter permease protein gene; and/or
   c) a mutation in the peptide deformylase protein gene,
wherein, preferably, the mutant strain shows improved texturing properties as defined herein as compared to the mother strain and, even more preferably, the obtained Streptococcus thermophilus strain shows the same or improved texturing properties as defined herein as compared to strain DSM22933. Hence, the method of the present invention may comprise a further screening step (iv), i.e., screening for a mutant strain which shows improved texturing properties as defined herein as compared to the mother strain (e.g., which generates higher shear stress and/or viscosity than the mother strain) when used for fermenting milk. Preferably, the mother strain in (i) is the deposited strain DSM22587.

Streptococcus thermophilus strains as defined in the present invention can also be generated by site directed mutagenesis, see step (ii) above. Oligonucleotides carrying the mutated nucleotide within the branched chain amino acid transport ATP-binding protein LivG (TC 3.A.1.4.1) and/or the ABC transporter permease protein and/or the peptide deformylase protein are used to amplify a specific DNA fragment by PCR. The PCR fragment carrying the desired mutation(s) is cloned into a vector plasmid and transformed into the S. thermophilus target strain, and the mutation is integrated into the chromosome and exchanging the wild type protein region by recombination. Isolation of strains is done as above. Hence, step (iii) above can also be the screening for a mutant strain comprising at least one, preferably two and, more preferably all three of the following amino acids and/or nucleotides in the following positions:
a) a Valine at a position corresponding to position 169 of SEQ ID NO.: 12 and/or a T at a position corresponding to position 506 of SEQ ID NO.: 11 (branched chain amino acid transport ATP-binding protein LivG (TC 3.A.1.4.1) gene);
b) a Phenylalanine at a position corresponding to position 190 of SEQ ID NO.: 4 and/or a T at a position corresponding to position 568 of SEQ ID NO.: 3 (ABC transporter permease protein gene); and/or
c) a Cysteine at a position corresponding to position 144 of SEQ ID NO.: 8 and/or a T at a position corresponding to position 430 of SEQ ID NO.: 7 (peptide deformylase protein gene).

In one embodiment, the mutation in the branched chain amino acid transport ATP-binding protein LivG (TC 3.A.1.4.1) gene (a) is a substitution of Glutamic acid to Valine at a position corresponding to position 169 of SEQ ID NO.: 12 and/or a mutation of nucleotide A to nucleotide T at a position corresponding to position 506 of SEQ ID NO.: 11. In one embodiment, the mutation in the ABC transporter permease protein gene (b) is a substitution of Leucine to Phenylalanine at a position corresponding to position 190 of SEQ ID NO.: 4 and/or a mutation of nucleotide C to nucleotide T at a position corresponding to position 568 of SEQ ID NO.: 3. In another preferred embodiment, the mutation in the peptide deformylase protein gene (c) is a substitution of Arginine to Cysteine at a position corresponding to position 144 of SEQ ID NO.: 8 and/or a mutation of nucleotide C to nucleotide T at a position corresponding to position 430 of SEQ ID NO.: 7.

Further, the present invention provides a method for manufacturing a Streptococcus thermophilus strain which shows improved texturing properties as defined herein as compared to the mother strain (e.g., which generates higher shear stress and/or viscosity than the mother strain) when the bacteria are used for fermenting milk, comprising the following steps:
a) providing a lactic acid bacterial strain as the mother strain;
b) exposing the mother strain to a bacteriophage which is able to lyse the mother strain;
c) isolating a mutant strain of the mother strain, which mutant strain is not lysed by the bacteriophage and has one, two or three mutations selected from the group consisting of:
   i. a mutation in the branched chain amino acid transport ATP-binding protein LivG (TC 3.A.1.4.1) gene;
   ii. a mutation in the ABC transporter permease protein gene; and/or
   iii. a mutation in the peptide deformylase protein gene; and
e) screening for a mutant strain which shows improved texturing properties as defined herein as compared to the mother strain (e.g., which generates higher shear stress and/or viscosity than the mother strain) when used for fermenting milk.

Preferably, the method of the present invention as described above comprises the step of incubating the exposed bacterial cells in a growth medium before step c) as described above. Preferably, the mother strain is strain DSM22587. Preferably, the bacteriophage able to lyse the mother strain of step b) above is DSM 24022.

In one embodiment, the (i) mutation in the branched chain amino acid transport ATP-binding protein LivG (TC 3.A.1.4.1) gene is a substitution of Glutamic acid to Valine at a position corresponding to position 169 of SEQ ID NO.: 12 and/or a mutation of nucleotide A to nucleotide T at a position corresponding to position 506 of SEQ ID NO.: 11. In one embodiment, the mutation in the ABC transporter permease protein gene is a substitution of Leucine to Phenylalanine at a position corresponding to position 190 of SEQ ID NO.: 4 and/or a mutation of nucleotide C to nucleotide T at a position corresponding to position 568 of SEQ ID NO.: 3. In one embodiment, the mutation in the peptide deformylase protein gene is a substitution of Arginine to Cysteine at a position corresponding position 144 of SEQ ID NO.: 8 and/or a mutation of nucleotide C to nucleotide T at a position corresponding to position 430 of SEQ ID NO.: 7.

Step d) above can also comprise introducing at least one, preferably two and, more preferably all three of the following amino acids/nucleotides at the following positions:
a) a Valine at a position corresponding to position 169 of SEQ ID NO.: 12 and/or a T at a position corresponding to position 506 of SEQ ID NO.: 11 (branched chain amino acid transport ATP-binding protein LivG (TC 3.A.1.4.1) gene);
b) a Phenylalanine at a position corresponding to position 190 of SEQ ID NO.: 4 and/or a T at a position corresponding to position 568 of SEQ ID NO.: 3 (ABC transporter permease protein gene); and/or
c) a Cysteine at a position corresponding to position 144 of SEQ ID NO.: 8 and/or a T at a position corresponding to position 430 of SEQ ID NO.: 7 (peptide deformylase protein gene).

Step d) above (e.g., introducing at least one, preferably two and, more preferably all three of the described mutations) can be performed by exposing the mother strain to any conventionally used mutagenizing treatment including treatment with a chemical mutagen or UV light and/or by site directed mutagenesis as defined herein.

Methods for determining the texture of fermented products such as dairy products include measuring the shear stress or viscosity (e.g., with the viscosity pipette test as described herein) of the fermented product and are readily available and known in the art and described and exemplified herein.

In one embodiment the Streptococcus thermophilus strain of the invention and/or the Streptococcus thermophilus strain directly obtained by the method described above generates a shear stress that is at least 1% improved when compared to its mother strain, such as 2%, 3%, 4%, 5%, 6%, 7%, 8%, 9%, 10%, 15%, 20% or more when compared to its mother strain. The shear stress characteristics of the Streptococcus thermophilus strain of the invention and/or the Streptococcus thermophilus strain directly obtained by the method described above can be measured in mixed cultures, as described in Example 5. In addition, the shear stress characteristics of DSM2293 can be measured for the strain on its own, as described above.

In one embodiment the Streptococcus thermophilus strain of the invention and/or the Streptococcus thermophilus strain directly obtained by the method described above generates a shear stress that is at least 1% improved when compared to its mother strain, such as 2%, 3%, 4%, 5%, 6%, 7%, 8%, 9%, 10%, 15%, 20% or more when compared to its mother strain when measured at 300 1/s (Pa) after about less than 5 hours (until pH of approx. 4.60) of growth in milk (3.6% protein and 1.5% or 3% fat) at 43° C. when inoculated in an amount of 0.02% FD-DVS starter culture.

In one embodiment the Streptococcus thermophilus strain of the invention and/or the Streptococcus thermophilus strain directly obtained by the method described above generates an efflux time of milk coagulated with that strain from the pipette that is at least 1% improved when compared to its mother strain, such as 2%, 3%, 4%, 5%, 6%, 7%, 8%, 9%, 10%, 15%, 20% or more when compared to its mother strain. The pipette viscosity test can be performed as described in Example 2. The pipette viscosity test can be performed as a mixed culture, as described in Example 3.

By “texture” or “mouthfeel” are meant the product's physical and chemical interaction in the mouth.

Use of the Streptococcus thermophilus Strains of the Present Invention and/or the Compositions of the Present Invention

One aspect of the present invention relates to the use of the Streptococcus thermophilus strains and/or compositions of the present invention for the manufacture of a fermented product. Again, the fermented product may be a dairy product.

Any combination of the above-described elements, aspects and embodiments in all possible variations thereof is encompassed by the invention unless otherwise indicated herein or otherwise clearly contradicted by context. Embodiments of the present invention are described below, by way of examples only.

### EXAMPLES

### Example 1: Phage Resistant Mutant of Streptococcus thermophilus Strain DSM22587

Phage resistant mutants were generated from the mother strain DSM22587 on M17-2% lactose agar plates with 10 mM MgCl₂/CaCl₂ after plating 0.1 mL of an M17-2% lactose overnight culture of DSM22587 together with 0.1 mL of phage DSM24022 containing 1×10⁸ phage particles per mL and incubation overnight at 37° C. Among several mutants, the strain DSM22933, was three times colony purified and retested in plaque on M17 lactose agar plates at 37° C. test using phage DSM24022 for phage challenge, and phage resistance was confirmed (no single plaques observed in plaque test). DSM22933 was also tested in milk acidification test showing an acidification activity comparable to the mother strain.

The genome of DSM22933 was sequenced at Chr. Hansen A/S as described by Agersø et al., 2018. In brief, total DNA was purified and used to prepare a 250-bp paired-end library for genome sequencing using Illumina MiSeq system. The sequence reads were subjected to quality trimming (Phred score <25) and assembled into contigs using the de novo assembly algorithm in CLC Genomics Workbench, version 10.1.1 (CLC bio, Qiagen Bioinformatics). The resulting genome assembly was filtered by removing contigs with coverage of <15× and/or <20% of the median coverage of the assembly. The consensus sequence of the remaining contigs was exported in FASTA format, which is referred to as the draft genome sequence, and used in the subsequent sequence analysis.

TABLE 1
Mutations identified in DSM22933 as compared to its mother strain DSM22587.

| | Gene | SEQ ID | Protein | SEQ ID |
|---|---|---|---|---|
| Name | mutation | No. | mutation | No. |
| Branched chain amino acid transport ATP- | A506T | 11 | Glu169Val | 12 |
| binding protein LivG (TC 3.A.1.4.1) gene | | | | |
| ABC transporter permease protein gene | C568T | 3 | Leu190Phe | 4 |
| Peptide deformylase protein gene | C430T | 7 | Arg144Cys | 8 |

### Example 2: Viscosity Pipette Test of DSM22933 as a Single Strain

Viscosity was measured by pipette test. In this test, the efflux time from a volumetric pipette is determined. A longer efflux time corresponds to higher viscosity. Coagulated milk was made from 200 mL skimmed milk inoculated with 1% of the bacterial strains to be tested (from an overnight culture grown in skimmed milk at 37° C.), and incubated for 20 h at 42° C. The viscosity of the coagulated milk was measured with a 25 mL volumetric pipette where the efflux time of said coagulated milk from the pipette was measured in triplicates. The coagulated milk is stirred carefully with a spoon to homogenize. The 25 mL volumetric pipette is then filled and the time to empty the pipette by gravity force is measured. The time it takes to empty 25 mL of coagulated milk from the pipette is noted as seconds.

DSM22933 provides a higher viscosity than its mother strain DSM22587 as it yields a 25% increase in efflux time measured by pipette test shown in the table below.

TABLE 2
Pipette viscosity test results for DSM22933
compared to its mother strain DSM22587.

| | Efflux | Efflux | Efflux | Average | |
|---|---|---|---|---|---|
| | time | time | time | efflux | |
| | sample 1 | sample 2 | sample 3 | time | Standard |
| | (s) | (s) | (s) | (s) | deviation |
| DSM22587 | 19 | 20 | 20 | 20 | 0.58 |
| DSM22933 | 24 | 26 | 25 | 25 | 1.00 |

### Example 3: Viscosity Pipette Test of DSM22933 in a Mixed Culture

Pipette tests were conducted as described in Example 2, with the exception that the overnight culture and the final incubation were made at 37° C. Fermentation with mixed cultures of 0.9% DSM22933 or DSM22587, and 0.1% Lactobacillus delbrueckii subsp. bulgaricus strain DSM19251 and 1.0% yeast extract were allowed to reach final pH 3.8.

The strain of the invention DSM22933 also shows improved viscosity in a mixed culture with L. bulgaricus strain DSM19251 and yeast as apparent from the table below.

TABLE 3
Pipette viscosity test results for DSM22933 in a mixed culture.

| | Efflux | Efflux | Efflux | Average | |
|---|---|---|---|---|---|
| | time | time | time | efflux | |
| | sample 1 | sample 2 | sample 3 | time | Standard |
| | (s) | (s) | (s) | (s) | deviation |
| DSM22587 | 176 | 204 | 141 | 157 | 17.8 |
| DSM22933 | 227 | 204 | 156 | 196 | 36 |

### Example 4: Aspiration of Mini Yoghurts Made with DSM22933

Mini yogurts were made from with a milk base comprising 4.0% protein and 0.1% fat inoculated with 0.02% F-DVS starter culture. Texturizing property of DSM22933 and DSM24023 were evaluated by adding the strains individually into 22 different mixed cultures for comparison. The 22 mixed cultures all comprised different combinations of a S. thermophilus strain and a L. bulgaricus strain. Aspiration was measured to check for increased texture. The more negative the aspiration, the better texture of the mini yoghurts. The two strains (DSM22933 and DSM24023) are sister strains derived from the same mother strain DSM22587. DSM24023 does not comprise the three mutations as described for DSM22933 in Table 1 above.

Cultures comprising DSM22933 resulted in a higher need for pressure to aspirate the yoghurt (−1808 Pa), when looking at an average of the 22 mini yoghurts, as compared to cultures comprising DSM24023 (−1692 Pa).

### Example 5: Rheological Property of Yoghurt Made with DSM22933

Milk base 1 with 3.6% protein and 3% fat (MB1) or Milk base 2 with 3.6% protein and 1.5% fat (MB2) were each inoculated with 0.02% FD-DVS starter culture. Fermentation was conducted at 43° C. until pH 4.60. The set yoghurt was stored at 6° C. for 7 days. The rheological property shear stress was measured with rheometer at 13° C. on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707-300] s⁻¹ and γ′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ was chosen.

Fermentation using a culture with DSM22933 was compared to fermentation using a benchmark culture with a prior art strain DSM22589 without the mutations described in Table 1 above. Both cultures comprised a further S. thermophilus strain which is identical in the two cultures and a non-texturizing L. bulgaricus strain.

The culture comprising DSM22933 shows improved viscosity as compared to the benchmark as apparent from the two tables below. The results were obtained using 30% DSM22933 as compared to 50% DSM22589.

TABLE 4
Rheological property of yoghurt made
with Milk base 1 and DSM22933.

| | Shear stress at | Shear stress at |
|---|---|---|
| MB1 | 30 s⁻¹ (Pa) | 300 s⁻¹ (Pa) |
| Benchmark | 53 | 53 |
| Culture with DSM22933 | 65 | 59 |

TABLE 5
Rheological property of yoghurt made
with Milk base 2 and DSM22933.

| | Shear stress at | Shear stress at |
|---|---|---|
| MB2 | 30 s−1 (Pa) | 300 s−1 (Pa) |
| Benchmark | 44 | 44 |
| Culture with DSM22933 | 49 | 48 |

### Deposits and Expert Solutions

The applicant requests that a sample of the deposited microorganisms stated in the table below may only be made available to an expert approved by the applicant.

TABLE 6
Deposits made at a Depositary institution having acquired the status of
international depositary authority under the Budapest Treaty on the
International Recognition of the Deposit of Microorganisms for the
Purposes of Patent Procedure: Leibniz Institute DSMZ-
German Collection of Microorganisms and Cell Cultures
Inhoffenstr. 7B, D-38124 Braunschweig, Germany.

| Microorganisms | Accession No. | Deposit date |
|---|---|---|
| Streptococcus thermophilus | DSM 22933 | 2009 Sep. 8 |

TABLE 7
References for other microorganisms

| Other Microorganisms | Accession No. | Reference |
|---|---|---|
| Streptococcus thermophilus | DSM22587 | WO2011/026863 |
| Streptococcus thermophilus | DSM22589 | WO2007/095958 |
| Streptococcus thermophilus | DSM24023 | WO2011/092300 |
| Lactobacillus delbrueckii | DSM19251 | WO2011/026863 |
| subsp. Bulgaricus | | |
| Bacteriophage | DSM24022 | WO2011/092300 |

### REFERENCES

Agersø et al., (2018)
Broadbent et al. (2003) J. Dairy Sci., 86:407-423
EMBOSS: The European Molecular Biology Open Software Suite, Rice et al. (2000) Trends in Genetics 16:276-277
Needleman and Wunsch (1970) J. Mol. 25 Biol. 48: 443-453

1. A Streptococcus thermophilus strain having:
(i) a mutation in the branched chain amino acid transport ATP-binding protein LivG (TC 3.A.1.4.1) gene;
(ii) a mutation in the ABC transporter permease protein gene; and/or
(iii) a mutation in the peptide deformylase protein gene.

2. The Streptococcus thermophilus strain according to claim 1, wherein:
(i) The mutation in the branched chain amino acid transport ATP-binding protein LivG (TC 3.A.1.4.1) is a substitution of Glutamic acid to Valine at a position corresponding to position 169 of SEQ ID NO.: 12 and/or a mutation of nucleotide A to nucleotide T at a position corresponding to position 506 of SEQ ID NO.: 11;
(ii) The mutation in the ABC transporter permease protein is a substitution of Leucine to Phenylalanine at a position corresponding to position 190 of SEQ ID NO.: 4 and/or a mutation of nucleotide C to nucleotide T at a position corresponding to position 568 of SEQ ID NO.: 3; and/or
(iii) The mutation in the peptide deformylase protein is a substitution of Arginine to Cysteine at a position corresponding to position 144 of SEQ ID NO.: 8 and/or a mutation of nucleotide C to nucleotide T at a position corresponding to position 430 of SEQ ID NO.: 7.

3. The Streptococcus thermophilus strain according to claim 1, wherein said strain generates higher shear stress and/or higher efflux time in a viscosity pipette test than a strain which does not have mutations (i), (ii) and/or (iii) as defined in claim 1, when used for fermenting milk.

4. The Streptococcus thermophilus strain according to claim 3, wherein said strain is derived from a mother strain which does not have mutations (i), (ii) and/or (iii) as defined in claim 1.

5. The Streptococcus thermophilus strain according to claim 1, wherein said strain is a phage resistant mutant from a strain which does not have mutations (i), (ii) and/or (iii) as defined in claim 1.

6. The Streptococcus thermophilus strain according to claim 3, wherein the strain which does not have mutations (i), (ii) and/or (iii) as defined in claim 1 is strain DSM22587.

7. The Streptococcus thermophilus strain according to claim 1, wherein the Streptococcus thermophilus strain is resistant to phage DSM24022.

8. The Streptococcus thermophilus strain according to claim 1, wherein the Streptococcus thermophilus strain is DSM22933 or a mutant or variant thereof.

9. The Streptococcus thermophilus strain according to claim 8, wherein the mutant or variant shows the same or similar texturing properties as DSM22933.

10. A composition comprising a Streptococcus thermophilus strain of claim 1.

11. The composition of claim 10 further comprising a strain belonging to Lactobacillus delbrueckii subsp bulgaricus.

12. The composition according to claim 10, wherein the composition is a starter culture, preferably in a frozen, spray-dried, freeze-dried, vacuum-dried, air dried, tray dried or liquid form.

13. A method of producing a fermented product comprising fermenting a milk substrate with the Streptococcus thermophilus strain according to claim 1.

14. A fermented product obtainable by the method according to claim 13.

15. The fermented product according to claim 14, wherein the product is a dairy product, preferably yoghurt, kefir, sour cream, quark or cheese.

16. A method for manufacturing a Streptococcus thermophilus strain with improved texturing properties as compared to the mother strain when the bacteria are used for fermenting milk, comprising the steps:
a) providing a lactic acid bacterial strain as the mother strain;
b) exposing the mother strain to a bacteriophage which is able to lyse the mother strain;
c) isolating a mutant strain of the mother strain, which mutant strain is not lysed by the bacteriophage and has one, two or three mutations selected from the group consisting of
i. a mutation in the branched chain amino acid transport ATP-binding protein LivG (TC 3.A.1.4.1) gene;
ii. a mutation in the ABC transporter permease protein gene; and
iii. a mutation in the peptide deformylase protein gene; and
d) screening for a mutant which shows improved texturing properties as compared to the mother strain when used for fermenting milk.

17. (canceled)
